(19) United States
(12) Patent Application Publication
(10) Pub. No.: US 2018/0306790 A1
(43) Pub. Date: Oct. 25, 2018

(54) USE OF EB1 AS A BIOMARKER OF DRUG RESPONSE

(71) Applicant: BASILEA PHARMACEUTICA INTERNATIONAL AG, Basel (CH)

(72) Inventors: Diane BRAGUER, Marseille (FR); Raphael BERGES, Marseille (FR); Stephane HONORE, Marseille (FR); Heidi LANE, Basel (CH); Felix BACHMANN, Basel (CH)

(21) Appl. No.: 15/769,137

(22) PCT Filed: Oct. 24, 2016
(86) PCT No.: PCT/EP2016/075542

§ 371 Date: Apr. 18, 2018

(30) Foreign Application Priority Data
Oct. 22, 2015 (EP) 15191127.8

(51) Int. Cl. G01N 33/574; G16H 50/30; A61K 31/4245

(52) U.S. Cl. CPC G01N 33/57407; G01N 33/57484; G01N 2800/56; A61K 31/4245; G01N 2800/52; G16H 50/30

(57) ABSTRACT

The present invention provides use of EB1 as a biomarker for predicting the response of a brain neoplasm to a compound of formula (I) wherein R represents phenyl or pyridinyl; wherein phenyl is optionally substituted by one or two substituents independently selected from lower alkyl, lower alkoxy, amino, acetylamino, halogen and nitro; and wherein pyridinyl is optionally substituted by amino or halogen; R¹ represents hydrogen or cyano-lower alkyl; or a pharmaceutically acceptable derivative thereof, and wherein the prefix lower denotes a radical having up to and including a maximum of 4 carbon atoms; in particular wherein a higher level of EB1 in the sample from the subject relative to a standard value or set of standard values predicts sensitivity of the brain neoplasm to the compound of formula I or pharmaceutically acceptable derivative thereof. The invention also provides methods of treatment and kits for use according to the invention.

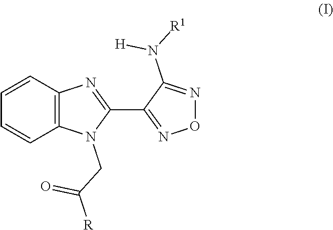

The present invention relates to use of EB1 as a biomarker for predicting the response of a cancer, in particular a brain neoplasm, to a compound of formula I as described below, such as 3-(4-{1-[2-(4-amino-phenyl)-2-oxo-ethyl]-1H-benzoimidazol-2-yl}-furazan-3-ylamino)-propionitrile (BAL27862) and derivatives thereof. In other aspects it relates to methods and kits.

Microtubules are one of the components of the cell cytoskeleton and are composed of heterodimers of alpha and beta tubulin. Microtubule targeting agents (MTAs) are among the most effective cytotoxic chemotherapeutic agents having a broad spectrum of activity. They are used for example in the treatment of hematologic malignancies and solid tumors, such as lung cancer, breast and prostate cancer. However, the use for treatment of glioblastoma is restricted due to the blood-brain barrier which does not allow most of the clinically relevant MTAs to cross into the brain where the tumor is located.

Resistance to MTAs can be either inherent or can be acquired after exposure to such agents. Such resistance therefore impacts patient survival rates, as well as choices of treatment regimes. Several potential mechanisms of resistance have been identified. This can include defects in the microtubule targets itself or in microtubule-associated proteins known to alter its biological properties and therefore responsiveness towards MTAs. Furthermore, defects in other cell proteins have been suggested to be associated with resistance to certain microtubule targeting agents, such as overexpression of efflux transporters which actively pump the drug out of the tumor cells. The standard oncology treatments have very often incomplete and temporary effects that only shrink the bulky tumor and the residual tumor tends to relapse mainly due to the multiple resistant mechanisms existing in small subpopulations of cancer stem cells (CSC). CSCs are long-lived with the ability of extensive self-renewal and tumor formation and recolonization.

Glioblastomas (GBMs) are the most common and most aggressive brain tumors among gliomas in adults. Despite progress, effective therapies are limited, especially in the recurrent setting. GBM tumors cause death due to rapid growth and a highly invasive behaviour within the whole brain. There is now compelling evidence that the majority of malignant cells are generated from CSCs, which are able to maintain and grow the tumor due to their capacity of self-renewal and resistance to chemo- and radiotherapy (Reya T., Stem cells, cancer, and cancer stem cells, Nature. 2001; 414:105-111; Pardal R., Applying the principles of stem-cell biology to cancer, Nat. Rev. Cancer. 2002; 3:895-902). The identification of CSC in brain tumors provides a powerful tool to investigate the development of malignant neuronal- or glia-derived tumor cells and to develop therapies to target these cells.

A recognized hallmark of CSCs responsible for tumor progression, maintenance and recurrence is their ability to migrate (Ortensi B., Cancer stem cell contribution to glioblastoma invasiveness, Stem Cell Research & Therapy. 2013; 4(1):18). This is an essential property for their infiltrative and also metastasizing behaviour which, clinically, makes complete tumor resection nearly impossible. Recent experimental data clearly shows that GBM CSCs are responsible for GBM invasiveness (Ortensi B., Cancer stem cell contribution to glioblastoma invasiveness, Stem Cell Research & Therapy. 2013; 4(1):18). GBM cells enriched for the stem cell marker CD133 either derived from human primary GBMs, GBM xenografts or GBM cell lines display greater migratory and invasive potential in vitro and in vivo when compared with matched CD133-negative GBM tumor cells (Yu S., Enhanced invasion in vitro and the distribution patterns in vivo of CD133+ glioma stem cells. Chin Med J. 2011; 124:2599-2604; Annabi B., Modulation of invasive properties of CD133+ glioblastoma stem cells: a role for MT1-MMP in bioactive lysophospholipid signaling. Mol Carcinog. 2009; 48:910-919). Furthermore, it has been demonstrated that pro-invasive genes are upregulated in CSCs and, hence, proteins which are involved in the process of migration and invasion are overexpressed (e.g. disintegrins, metallo-proteinases) (Ortensi B., Cancer stem cell contribution to glioblastoma invasiveness. Stem Cell Research & Therapy. 2013; 4(1):18).

Microtubule end-binding protein 1 (EB1) was originally discovered as a binding partner of the APC (adenomatous polyposis coli) tumor suppressor. EB1 is encoded by the MAPRE1 gene and is a member of the RP/EB family involved in the regulation of microtubule dynamics, cell polarity and chromosomal stability during mitosis (Dong X., Oncogenic function of microtubule end-binding protein 1 in breast cancer. J Pathol. 2010; 220(3):361-9; Wang Y., Overexpression of EB1 in human esophageal squamous cell carcinoma: promoting cellular growth probably by activating beta-catenin/TCF pathway. Cancer Res. 2005; 65:1304-1310). EB1/MAPRE1 has been assigned Human Gene Nomenclature Committee Identification number HGNC ID:6890 and Entrez Gene ID 22919. A sequence corresponding to human EB1 protein and nucleic acid is available via the National Center for Biotechnology Information (NCBI) reference number NM_012325 (FIG. 10, SEQ ID No. 1 & 2, NM_012325). Despite being an important binding partner of APC, the general role of EB1 in tumor formation has not been well established. However, recent reports suggest that EB1 itself has oncogenic functions. Expression studies have demonstrated that EB1 is overexpressed in breast cancer (Dong X., Oncogenic function of microtubule end-binding protein 1 in breast cancer, J Path. 2010; 220(3):361-9) gastric cancer, hepatocellular carcinoma, esophageal squamous cell carcinoma (Wang Y., Overexpression of EB1 in human esophageal squamous cell carcinoma: promoting cellular growth probably by activating beta-catenin/TCF pathway, Cancer Res. 2005; 65:1304-1310) and its expression level correlates with poor outcome including reduced progression-free survival (PFS) and overall survival (OS). This has also been shown in a more recently performed study in a cohort of 109 primary GBM patients (Berges R., End-binding 1 protein overexpression correlates with glioblastoma progression and sensitizes to Vinca-alkaloids in vitro and in viva Oncotarget. 2014; Vol. 5(24):12769-87). High EB1 expression was correlated to poor OS (p<0.001) and poor PFS (p<0.001) in the investigated GBM patients.

BAL27862 is described in WO 2004103994 for the treatment of neoplastic diseases and autoimmune diseases. Prodrugs thereof (e.g. BAL101553) are described in WO 2011012577.

It has now surprisingly been found that the presence of EB1 is associated with increased sensitivity of GBM CSCs to BAL101553 treatment.

In a first aspect the invention provides use of EB1 as a biomarker for predicting the response of a brain neoplasm to a compound of formula I

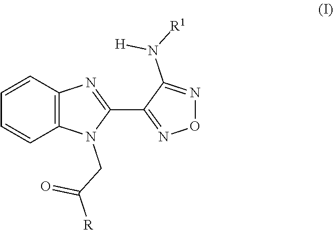

wherein

R represents phenyl or pyridinyl;

wherein phenyl is optionally substituted by one or two substituents independently selected from lower alkyl, lower alkoxy, amino, acetylamino, halogen and nitro;

and wherein pyridinyl is optionally substituted by amino or halogen;

R¹ represents hydrogen or cyano-lower alkyl;

or a pharmaceutically acceptable derivative thereof;

and wherein the prefix lower denotes a radical having up to and including a maximum of 4 carbon atoms.

Although compounds of formula I are microtubule destabilisers, they have a different effect on the phenotype of cells compared to other microtubule targeting agents, including other microtubule destabilisers. In addition they affect microtubule biology in a different manner than conventional microtubule targeting agents and consequently have potent activity in tumor models which are resistant to conventional microtubule-targeting agents. See the Examples of WO 2012098208, WO 2012098207, WO 2012098203, WO 2012113802 and WO 2012130887. Thus, predictions cannot be made from information about conventional microtubule targeting agents concerning if, or how, expression of particular genes is involved in the action of compounds of formula I.

Preferably the response is to treatment, i.e. to treatment with the compound of formula I or pharmaceutically acceptable derivatives thereof.

Preferably the response is the response of CSCs of the brain neoplasm to the compound of formula I or pharmaceutically acceptable derivative thereof.

In one embodiment a higher level of EB1 in a sample taken from a subject relative to a standard value or set of standard values predicts sensitivity of the brain neoplasm, in particular the sensitivity of CSCs in the brain neoplasm, to the compound of formula I or pharmaceutically acceptable derivative thereof.

In a further embodiment a higher level of EB1 in the CSCs in a sample taken from a subject relative to a standard value or set of standard values predicts sensitivity of the brain neoplasm, in particular the sensitivity of CSCs in the brain neoplasm, to the compound of formula I or pharmaceutically acceptable derivative thereof.

In a further embodiment a lower level of EB1 in a sample taken from a subject relative to a standard value or set of standard values predicts resistance of the brain neoplasm, in particular the resistance of CSCs in the brain neoplasm, to the compound of formula I or pharmaceutically acceptable derivative thereof.

In a further embodiment a lower level of EB1 in the CSCs in a sample from a subject relative to a standard value or set of standard values predicts resistance of the brain neoplasm, in particular the sensitivity of CSCs in the brain neoplasm, to the compound of formula I or pharmaceutically acceptable derivative thereof.

EB1 is measured ex vivo in samples taken from the subject.

Preferably the brain neoplasm is glioblastoma multiforme.

Preferably the compounds of formula I are BAL27862 or its prodrug BAL101533.

In a further embodiment the invention provides use of EB1 as a biomarker for predicting the response of glioblastoma multiforme to BAL27862 or BAL101533;

wherein the biomarker EB1 is measured ex vivo in a sample taken from a subject;

wherein a higher level of EB1 in the sample from the subject relative to a standard value or set of standard values predicts sensitivity of the glioblastoma multiforme to BAL27862 or BAL101533.

In a further embodiment the invention provides use of EB1 as a biomarker for predicting the response of glioblastoma multiforme to BAL27862 or BAL101533;

wherein the biomarker EB1 is measured ex vivo in a sample taken from a subject;

wherein the level of EB1 is measured in the CSCs in the sample, preferably in CSCs which express CD133 and/or A2B5;

wherein a higher level of EB1 in the CSCs in the sample from the subject relative to a standard value or set of standard values predicts sensitivity of the glioblastoma multiforme to BAL27862 or BAL101533.

In a further aspect of the invention provides a method for predicting the response to treatment of a brain neoplasm in a subject by administration of a compound of formula I, said method comprising the steps of:

a) measuring the level of the EB1 in a sample obtained from the subject to obtain a value or values representing this level; and

b) comparing the value or values of the levels from step a) with a standard value or a set of standard values which comparison is predictive of responsiveness to the compound of formula I.

The above embodiments apply to this aspect of the invention. In particular, in one embodiment the invention provides a method for predicting the response to treatment of glioblastoma multiforme in a human subject by administration of BAL27862 or BAL101533, said method comprising the steps of:

a) measuring ex vivo the level of the EB1 in a sample obtained from the human subject to obtain a value or values representing this level; and

b) comparing the value or values of the levels from step a) with a standard value or a set of standard values, which comparison is predictive of responsiveness to BAL27862 or BAL101533;

and wherein a higher level of EB1 in the sample from the human subject relative to a standard value or set of standard values predicts sensitivity of the glioblastoma multiforme to BAL27862 or BAL101533.

In a further embodiment the invention provides a method for predicting the response to treatment of glioblastoma multiforme in a human subject by administration of BAL27862 or BAL101533, said method comprising the steps of:

a) measuring ex vivo the level of the EB1 in the CSCs, preferably CSCs which express CD133 and/or A2B5, in a sample obtained from the human subject to obtain a value or values representing this level;

b) comparing the value or values of the levels from step a) with a standard value or a set of standard values, which comparison is predictive of responsiveness to BAL27862 or BAL101533;

and wherein a higher level of EB1 in the CSCs in the sample from the human subject relative to a standard value or set of standard values predicts sensitivity of the glioblastoma multiforme to BAL27862 or BAL101533.

In a further aspect the invention provides a compound of formula I or pharmaceutically acceptable derivative thereof for use in the treatment of a brain neoplasm, comprising measuring the level of EB1 in a sample from a subject to obtain a value or values representing this level, and treating the subject with the compound of formula I or a pharmaceutically acceptable derivative thereof, if the level of EB1 is higher than a standard value or set of standard values.

The above embodiments apply to this aspect of the invention. In particular, in one embodiment the invention provides BAL27862 or BAL101533 for use in the treatment of glioblastoma multiforme, comprising measuring ex vivo the level of EB1 in a sample from a taken from a human subject to obtain a value or values representing this level, and treating the human subject with BAL27862 or BAL101533, if the level of EB1 is higher than a standard value or set of standard values.

In a further embodiment the invention provides BAL27862 or BAL101533 for use in the treatment of glioblastoma multiforme, comprising measuring ex vivo the level of EB1 in the CSCs, preferably CSCs which express CD133 and/or A2B5, in a sample from a taken from a human subject to obtain a value or values representing this level, and treating the human subject with BAL27862 or BAL101533 if the level of EB1 in the CSCs in the sample is higher than a standard value or set of standard values.

In a further aspect the invention provides use of a compound of formula I or pharmaceutically acceptable derivative thereof in the manufacture of a medicament for the treatment of a brain neoplasm, comprising measuring the level of EB1 in a sample from a subject to obtain a value or values representing this level, and treating the subject with a compound of formula I or a pharmaceutically acceptable derivative thereof, if the level of EB1 is higher than a standard value or set of standard values.

The above embodiments apply to this aspect of the invention. In particular, in one embodiment the invention provides use of BAL27862 or BAL101533 in the manufacture of a medicament for the treatment of glioblastoma multiforme, comprising measuring ex vivo the level of EB1 in a sample taken from a human subject to obtain a value or values representing this level, and treating the human subject with BAL27862 or BAL101533, if the level of EB1 is higher than a standard value or set of standard values.

In a further embodiment the invention provides use of BAL27862 or BAL101533 in the manufacture of a medicament for the treatment of glioblastoma multiforme, comprising measuring ex vivo a level of EB1 in the CSCs, preferably CSCs which express CD133 and/or A2B5, in a sample from a taken from a human subject to obtain a value or values representing this level, and treating the human subject with BAL27862 or BAL101533 if the level of EB1 in the CSCs in the sample is higher than a standard value or set of standard values.

In a further aspect the invention provides a method of treating a brain neoplasm in a subject in need thereof, said method comprising

a) obtaining a sample of biologic material from the body of said subject;

b) determining the level of the EB1 in said sample; and

c) treating the subject with a compound of formula I or a pharmaceutically acceptable derivative thereof, if the level of EB1 in said sample is higher than a standard value or set of standard values.

The above embodiments apply to this aspect of the invention. In particular, in one embodiment the invention provides a method of treating glioblastoma multiforme in a human subject in need thereof, said method comprising

a) obtaining a sample of biologic material from the body of said human subject;

b) determining the level of the EB1 in said sample; and

c) treating the human subject with BAL27862 or BAL101533, if the level of EB1 in said sample is higher than a standard value or set of standard values.

In a further embodiment the invention provides a method of treating glioblastoma multiforme in a human subject in need thereof, said method comprising

a) obtaining a sample of biologic material from the body of said human subject;

b) determining the level of the EB1 in the CSCs in said sample, preferably CSCs which express CD133 and/or A2B5; and

c) treating the human subject with BAL27862 or BAL101533, if the level of EB1 the CSCs in said sample is higher than a standard value or set of standard values.

In a further aspect the invention provides a kit for predicting the response of a brain neoplasm to a compound of formula I or a pharmaceutically acceptable derivative thereof, comprising reagents necessary for measuring a level of EB1 in a sample and preferably further comprising a comparator module which comprises a standard value or set of standard values to which the level of EB1 in the sample is compared. In a preferred embodiment the kit includes BAL27862 and/or BAL101533

In a further aspect the invention provides a device for predicting the response of a brain neoplasm to a compound of formula I or a pharmaceutically acceptable derivative thereof, comprising reagents necessary for measuring a level of EB1 in a sample and preferably further comprising a comparator module which comprises a standard value or set of standard values to which the level of EB1 in the sample is compared. In a preferred embodiment the device includes BAL27862 and/or BAL101533.

In a preferred embodiment, the reagents in the kit or device comprise a capture reagent comprising a detector for EB1, and a detector reagent. Especially preferably the capture reagent is an antibody.

In further preferred embodiments, the kit or device comprise reagents necessary for measuring a level of EB1 in a sample and reagents necessary for identifying and/or capturing CSCs.

Also preferably, the brain neoplasm is predicted to be sensitive to treatment with said compound when the level of EB1 is higher relative to a standard value or set of standard values. In preferred embodiments, the comparator module represents instructions for use of the kit or device respectively. In an alternative embodiment the comparator module is in the form of a display device.

Embodiments of the present invention will now be described by way of example with reference to the accompanying figures. The invention however is not to be understood as limited to these embodiments.

### BRIEF DESCRIPTION OF THE FIGURES

FIG. 1 shows EB1 expression levels in GBM6 and GBM9 GBM CSCs.

FIG. 2 shows inhibition of in vitro cell migration in GBM6 and GBM9 CSCs. Quantification is expressed as a percentage of migrating cells relative to 100% of control cells for GBM6 and GBM9, respectively. The results from at least three independent experiments are shown. Asterisks indicate statistically significant differences between groups as indicated: **: p<0.005, ***: p<0.001

FIG. 3 shows analysis of stable GFP-expressing GBM6 CSCs with EB1 expression levels suppressed by shRNA.

FIG. 4 shows the effect of EB1 downregulation in GBM6 CSCs on the anti-migratory effect of BAL27862 at non-cytotoxic concentration of 6 nM. The results from at least three independent experiments are shown. Asterisks indicate statistically significant differences to control or between groups: p<0.05.

FIG. 5 shows the effect of BAL27862 on the clonogenic capacity of GBM6 CSCs in an EB1 expression-dependent manner. The percentage of colony formation was calculated by the number of colonies formed divided by the original number of cells seeded×100. The mean values and SEM of at least 3 independent experiments are shown. Asterisks indicate statistically significant differences to control or between groups as indicated: *: p<0.05, **: p<0.005.

FIG. 6 shows the effect of BAL27862 on the in vitro differentiation of GBM6 CSCs in an EB1-expression dependent manner. The percentage of A2B5 positive cells was calculated in relation to the cell count of the control groups. Graphs represent mean values and SEM of at least 3 independent experiments. Asterisks indicate statistically significant differences to untreated control (p<0.005), ns: not significant.

FIG. 7 shows the effect of BAL27862 on CSC differentiation in vitro in a EB1-dependent manner. The graph shows mean values and SEM of at least 3 independent experiments. Asterisks indicate statistically significant differences to untreated control (p<0.005), ns: not significant.

FIG. 8 shows BAL101553 activity on orthotopic GBM6 tumors in vivo on day 45. Black shading indicates tumor location and size.

FIG. 9 shows the effect of BAL101553 treatment on the proportion of undifferentiated CSCs in orthotopic GBM6 tumors.

FIG. 10 shows the amino acid sequence (FIG. 10A) (GenBank AAC09471—SEQ ID NO:1) and the nucleic acid sequence (FIG. 10B) (GenBank U24166—SEQ ID NO: 2) of human EB1.

FIG. 11 shows the expression of EB1 protein in three tumors derived from GBM10 tumors. Immunoblots of tumor extracts are shown with actin as a loading control and HeLa extracts derived from cells treated with non-targeting control (NTC) siRNA or EB1 siRNA confirming the specificity of the EB1 antibody.

FIG. 12 shows the expression of EB1 in extracellular vesicles (including exosomes) derived from the serum of GBM10 tumor-bearing mice. Immunoblots include HeLa extracts derived from cells treated with non-targeting control (NTC) siRNA or EB1 siRNA confirming the specificity of the EB1 antibody. CD9 immunoblots act as a marker of extracellular vesicles (EV) with exosomes derived from human serum from healthy donors (Hu) and HeLa extracts derived from cells treated with non-targeting control (NTC) siRNA included as CD9 positive and negative controls, respectively.

### DETAILED DESCRIPTION

Compounds of Formula I

Preferred compounds of formula I include those wherein R, Y and R¹ are defined as follows:

| R | Y | R¹ |
|---|---|---|
| 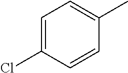 | O | H |
| 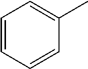 | NOH | H |
| 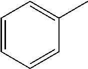 | NOMe | H |
| 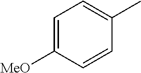 | O | H |
| 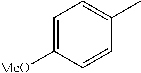 | NOH | H |
| 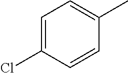 | NOH | H |
| 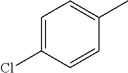 | NOMe | H |
| 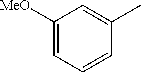 | O | H |
| 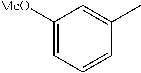 | NOH | H |
| 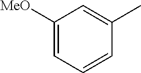 | NOMe | H |
| 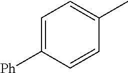 | O | H |
| 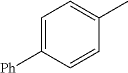 | NOH | H |
| 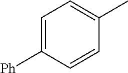 | NOMe | H |
| 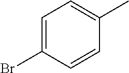 | O | H |
| 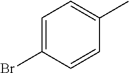 | NOMe | H |
| 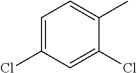 | O | H |
| 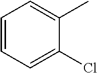 | O | H |
| 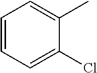 | NOH | H |
| 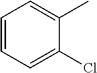 | NOMe | H |
| 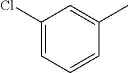 | O | H |
| 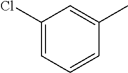 | NOH | H |
| 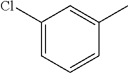 | NOMe | H |
| 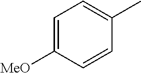 | NOMe | H |
| 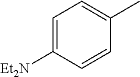 | O | H |
| 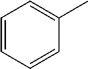 | O | Ac |
| 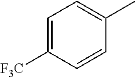 | O | H |
| 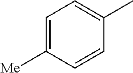 | O | H |
| 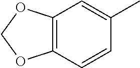 | O | H |
| 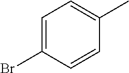 | O | CH₂CH₂CN |
| 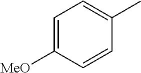 | O | CH₂CH₂CN |
| 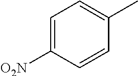 | O | H |
| 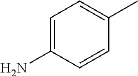 | O | H |
| 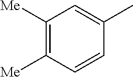 | O | CH₂CH₂CH₂OH |
| 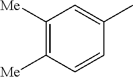 | O | H |
| 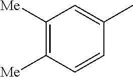 | O | CH₂CH₂CN |
| 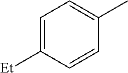 | O | H |
| 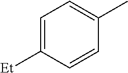 | O | CH₂CH₂CN |
| 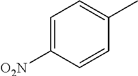 | O | CH₂CH₂CN |
| 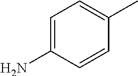 | O | CH₂CH₂CN |
| 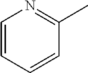 | O | H |
| 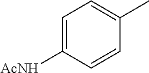 | O | H |
| 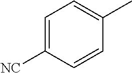 | O | H |
| 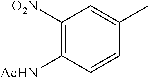 | O | H |
| 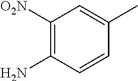 | O | H |
| 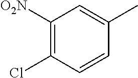 | O | H |
| 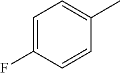 | O | H |
| 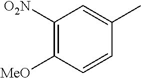 | O | H |
| 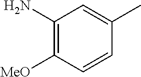 | O | CH₂CH₂CN |
| 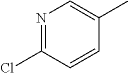 | O | H |
| 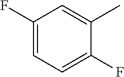 | O | H |
| 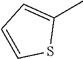 | O | H |
| 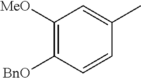 | O | H |
| 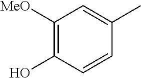 | O | H |
| 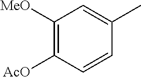 | O | H |
| 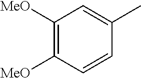 | O | H |
| 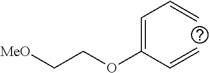 | O | H |
| 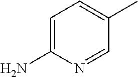 | O | H |
| 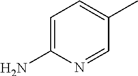 | O | CH₂CH₂CN |
| 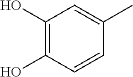 | O | H |
| 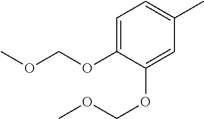 | O | H |
| 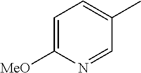 | O | CH₂CH₂CN |

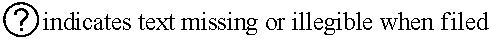

or pharmaceutically acceptable derivatives thereof.

Especially preferred are compounds wherein R, Y and R¹ are defined as follows:

| R | Y | R¹ |
|---|---|---|
| 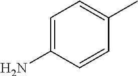 | O | H |
| 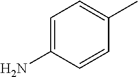 | O | CH₂CH₂CN |
| 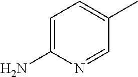 | O | H |
| 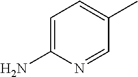 | O | CH₂CH₂CN |

or pharmaceutically acceptable derivatives thereof.

An especially preferred compound is BAL27862:

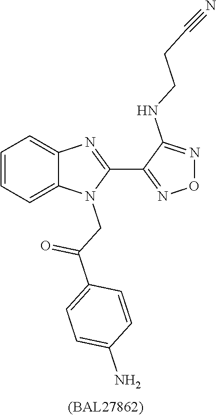 [SMILES: N#CCCNc1nonc1-c1nc2ccccc2n1CC(=O)c1ccc(N)cc1]

The term derivative or derivatives in the phrase “pharmaceutically acceptable derivative” or “pharmaceutically acceptable derivatives” of compounds of formula I relates to salts, solvates and complexes thereof and to solvates and complexes of salts thereof, as well as to pro-drugs, polymorphs, and isomers thereof (including optical, geometric and tautomeric isomers) and also salts of pro-drugs thereof. In a more preferred embodiment, it relates to salts and pro-drugs, as well as to salts of pro-drugs thereof.

Salts are preferably acid addition salts. Salts are formed, preferably with organic or inorganic acids, from compounds of formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

The compound according to the invention may be administered in the form of a pro-drug which is broken down in the human or animal body to give a compound of the formula I. Examples of pro-drugs include in vivo hydrolysable esters and amides of a compound of the formula I. Particular pro-drugs considered are ester and amides of naturally occurring amino acids and ester or amides of small peptides, in particular small peptides consisting of up to five, preferably two or three amino acids as well as esters and amides of pegylated hydroxy acids, preferably hydroxy acetic acid and lactic acid. Pro-drug esters are formed from the acid function of the amino acid or the C terminal of the peptide and suitable hydroxy group(s) in the compound of formula I. Pro-drug amides are formed from the amino function of the amino acid or the N terminal of the peptide and suitable carboxy group(s) in the compound of formula I, or from the acid function of the amino acid or the C terminal of the peptide and suitable amino group(s) in the compound of formula I. Particularly preferably the pro-drug amides are formed from the amino group(s) present within the R group of formula I.

More preferably, the pro-drug is an amide formed from an amino group present within the R group of the compound of formula I as defined above and the carboxy group of glycine, alanine or lysine.

Even more preferably the compound of formula I is in the form of a pro-drug selected from the compounds of formulae:

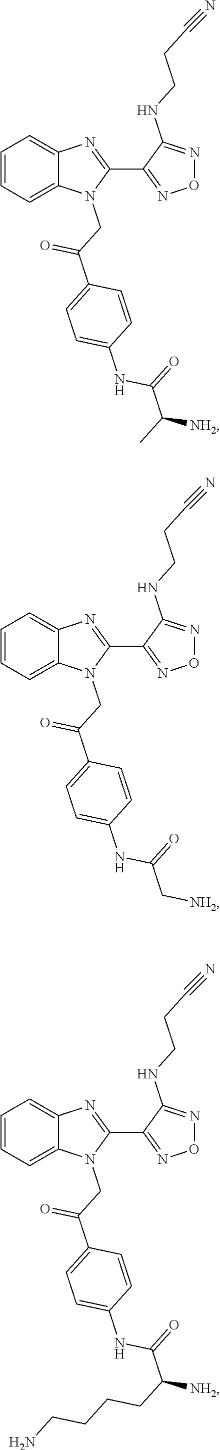 [SMILES: C[C@H](N)C(=O)Nc1ccc(C(=O)Cn2c(-c3nonc3NCCC#N)nc3ccccc32)cc1 ; N#CCCNc1nonc1-c1nc2ccccc2n1CC(=O)c1ccc(NC(=O)CN)cc1 ; N#CCCNc1nonc1-c1nc2ccccc2n1CC(=O)c1ccc(NC(=O)[C@@H](N)CCCCN)cc1]

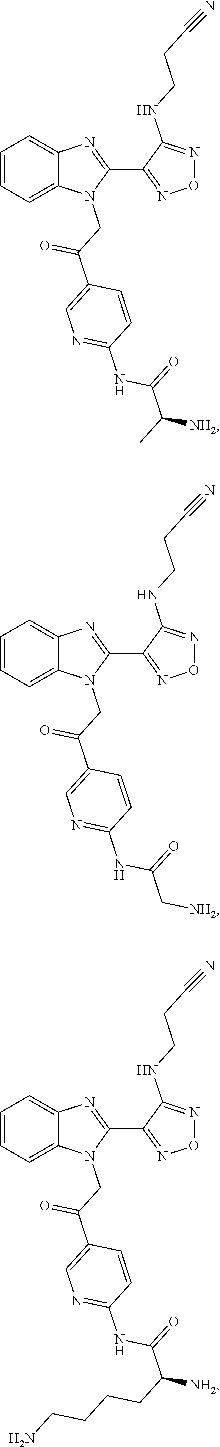 [SMILES: C[C@H](N)C(=O)Nc1ccc(C(=O)Cn2c(-c3nonc3NCCC#N)nc3ccccc32)cn1 ; N#CCCNc1nonc1-c1nc2ccccc2n1CC(=O)c1ccc(NC(=O)CN)nc1 ; N#CCCNc1nonc1-c1nc2ccccc2n1CC(=O)c1ccc(NC(=O)[C@@H](N)CCCCN)nc1]

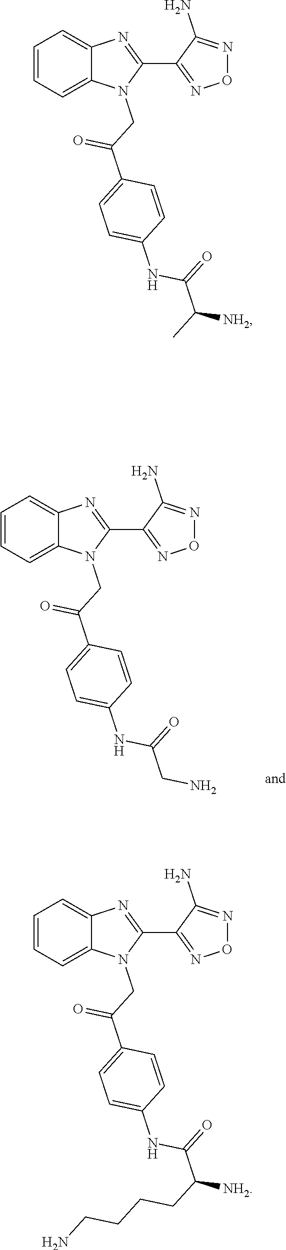 [SMILES: C[C@H](N)C(=O)Nc1ccc(C(=O)Cn2c(-c3nonc3N)nc3ccccc32)cc1 ; NCC(=O)Nc1ccc(C(=O)Cn2c(-c3nonc3N)nc3ccccc32)cc1 ; NCCCC[C@H](N)C(=O)Nc1ccc(C(=O)Cn2c(-c3nonc3N)nc3ccccc32)cc1]

In an especially preferred embodiment the compound of formula I according to the invention is in the form of the pro-drug BAL101553:

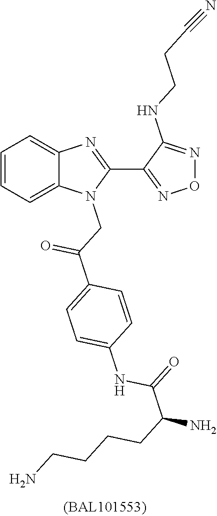 [SMILES: N#CCCNc1nonc1-c1nc2ccccc2n1CC(=O)c1ccc(NC(=O)[C@@H](N)CCCCN)cc1]

or a pharmaceutically acceptable salt thereof, preferably a hydrochloride salt, most preferably a dihydrochloride salt.

The prodrugs of the invention may be prepared as described for example in WO 2012/098207 on pages 37 to 39, which are hereby incorporated by reference.

Disease

Brain neoplasms, e.g. brain tumors, include but are not limited to glial- and non-glial-tumors, astrocytomas (incl. glioblastoma multiforme and unspecified gliomas), oligodendrogliomas, ependydomas, menigiomas, haemangioblastomas, acoustic neuromas, craniopharyngiomas, primary central nervous system lymphoma, germ cell tumors, pituitary tumors, pineal region tumors, primitive neuroectodermal tumors (PNET's), medullablastomas, haemangiopericytomas, spinal cord tumors including meningiomas, chordomas and genetically-driven brain neoplasms including neurofibromatosis, peripheral nerve sheath tumors and tuberous sclerosis. Preferably, brain neoplasm refers to glioblastomas (also referred to as glioblastoma multiforme).

Samples

The measurement of the level of EB1 is performed ex vivo on a sample of biological tissue derived from the subject. The sample may be any biological material separated from the body such as, for example, normal tissue, tumor tissue, cell lines, peripheral blood (including circulating tumor cells or CSCs), cerebrospinal fluid (including CSCs), lymph fluid, cell lysate, tissue lysate, urine and aspirates. Preferably the sample is derived from normal tissue, tumor tissue, cell lines, peripheral blood (including circulating tumor cells or CSCs) or cerebrospinal fluid (including CSCs). More preferably the sample is derived from tumor tissue or cancer stem cells derived from peripheral blood or cerebrospinal fluid. Even more preferably the sample is derived from tumor tissue or CSCs derived from peripheral blood. In one particularly preferred embodiment the sample is derived from tumor tissue. For example, the level of EB1 in cancer cells and/or CSCs may be measured in a fresh, frozen or formalin fixed/paraffin embedded tumor tissue sample. Alternatively, when the sample is derived from a body fluid, for example blood (e.g. peripheral blood), serum and plasma, urine, cerebrospinal fluid or saliva the level of EB1 may be measured in the extracellular vesicles in the sample.

The sample is pre-obtained from the subject before the sample is subjected to method steps involving measuring the level of the biomarker. Methods for taking a sample from a subject are well known in the art. Methods of removing a sample from a tumor may involve for example tumor resection or biopsy, for example by punch biopsy, core biopsy or aspiration fine needle biopsy, endoscopic biopsy, or surface biopsy.

Brain CSCs very often spread to different sites in the brain and form secondary lesions and metastases. To do so, they may invade their surroundings and thereby may also destroy structures supporting the blood-brain-barrier. As a consequence of this invasion process, they may gain access to the blood circulation system and therefore can be identified/purified from peripheral blood (Watkins, S. Disruption of astrocytes-vascular coupling and the blood-brain barrier by invading glioma cells, Nature Communications 5. 2014; Article number: 4196; Diaz, M., Transmigration of Neural Stem Cells across the blood-brain barrier induced by glioma cells. PLoS One. 2013; 8(4); Beauchesne P., Extra-Neural Metastases of Malignant Gliomas: Myth or Reality. Cancers. 2011; 3:461-477). A blood sample may be collected by venipuncture and further processed according to standard techniques. Circulating tumor cells and/or circulating CSCs may also be obtained from blood or cerebrospinal fluid based on, for example, size (e.g. ISET—Isolation by Size of Epithelial Tumor cells; by Cluster-chip based on the size of preformed cell clusters) or immunomagnetic cell enrichment (e.g. CellSearch®, Veridex, Raritan, N.J.; MACS®, Miltenyi Biotec, Germany; RosetteSep™, EasySep™ and RoboSep™, STEMCELL Technologies, France) or fluorescence activated cell sorting (FACS) (e.g. BD Stemflow Kit, BD-Biosciences, USA) or cell separation based on dielectric properties (ApoStream®, APOCELL, USA).

Brain tumor material, e.g. GBM material, may be obtained from patients undergoing surgery or tumor biopsy. Resected GBM tumor material may then be further processed by methods known in the art including, but not restricted to, placing the tumor material into a tube on ice containing neural stem cell (NSC) basal medium supplemented with 10-15% antibiotics (Penicilin/Streptomycin). The following procedure is given as an example. The GBM tumor samples are washed 2-3 times with sterile PBS/NSC basal medium to remove blood and debris. In a sterile Petri dish the washed tumor tissue is cut into small pieces and minced with a scalpel blade, transferred into a Falcon tube and trypsinized in a few mL's of pre-warmed 0.05% trypsin-EDTA for 10-15 minutes in a water bath at 37° C. After the incubation period, an equal volume of soybean trypsin inhibitor is added to stop the enzymatic trypsin reaction. The tumor cell suspension is pelleted down by centrifugation at 800 rpm (110 g) for 5 minutes. The supernatant is discarded and the pellet is resuspended in 1 mL of sterile NSC basal medium. The clumps are dissociated by gently pipetting up and down and the cell suspension is passed through a 40 micron cell strainer to remove small cell debris (Azari, H., Isolation and Expansion of Human Glioblastoma Multiforme Tumor Cells Using the Neurosphere Assay. J. Vis. Exp. 2011; October 30(56):e3633).

As an alternative approach, dissociation of the tumor tissue into a tumor cell suspension may also be performed using a commercially available kit including, but not restricted to, “The Brain Tumor Dissociation Kits” available from Miltenyi Biotec Inc., Auburn, Calif. 95602, USA.

It is known that brain tumours such as those associated with GBM release extracellular vesicles into the blood which may then circulate around the body (Arscott et al., Ionizing Radiation and Glioblastoma Exosomes: Implications in Tumor Biology and Cell Migration, Translational Oncology, 2006; 6:638-648). Extracellular vesicles include microvesicles and exosomes and are generally nanometer-sized membrane-derived vesicles. Extracellular vesicles contain molecular information (including protein and RNA) reflecting the cell of origin including hallmarks of tumor biology (Redzik et al., Glioblastoma extracellular vesicles: reservoirs of potential biomarkers. Pharmgenomics Pers. Med., 2014; 7:65-77; Capello et sl., Exosome levels in human body fluids: A tumor marker by themselves? Eur. J. Pharm. Sci., 2016; 96:93-98) and are accessible in body fluids such as blood, including serum and plasma, urine, cerebrospinal fluid and saliva. This provides an alternative method of determining whether EB1 is present in GBM tumors.

Extracellular vesicles can be isolated using a variety of protocols and commercially available kits including: a) differential ultracentrifugation (Thery et al., Isolation and characterization of exosomes from cell culture supernatants and biological fluids. Curr. Prot. Cell Biol., 2006; 3.22.1-3.22.29); b) chemical precipitation (e.g. Total Exosome Isolation Kits from Invitrogen, ThermoFischer, Waltham, Mass. USA; ExoQuick™ Exosome Precipitation Solution from SBI System Biosciences, Palo Alto, USA; Exo-Prep from HansaBioMed, Tallinn, Estonia); and c) immunoaffinity capture (Zarovni et al., Integrated isolation and quantitative analysis of exosome shuttled proteins and nucleic acids using immunocapture approaches. Methods, 2015; 87:46-58) using for example ELISA immunoplates (e.g. from HansaBioMed), Immunobeads (e.g. from HansaBioMed) and ExoCap™ from JSR Life Sciences, Leuven, Belgium. The level of EB1 nucleic acid (preferably RNA) and/or protein in the extracellular vesicles can be measured by standard techniques as described below.

Cancer Stem Cells (CSCs)

CSCs in a sample can be identified by methods known in the art including methods based on identifying CSC markers e.g. using immunohistochemistry or flow cytometry and methods based on selecting for properties that are characteristic of CSCs but not possessed by differentiated cancer cells, e.g. selecting for cells that have pluripotent properties and/or are able to propagate under in vitro culture conditions.

CSC markers that may be used to identify CSCs are ALDH1, CD24, CD44, CD90, CD133, Hedgehog-Gli activity, α6-integrin, ABCB5, β-catenin activity, CD26, CD29, CD166, LGR5, CD15, nestin, CD13, ABCG2, CD117, CD20, CD271, c-Met, CXCR4, Nodal-Activin, α2β1-integrin and Trop2, whilst CD15, CD90, CD133, α6-integrin, nestin, and A2B5 may be specific for CSCs in glioblastoma multiforme (Medema J P., Cancer stem cells: The challenges ahead, Nature Cell Biology. 2013; 15:338-344). Of particular interest for the present invention are CD133 and/or A2B5. (Tchoghandjian A et al., A2B5 cells from human glioblastoma have cancer stem cell properties. Brain Pathol. 2010; 1:211-21).

The following procedures are given as examples of identifying brain CSCs in a sample taken from a subject.

Brain tumor cell suspensions are seeded at 300′000 cells/well into previously poly-DL-ornithine-coated 6-well plates, in stem cell-permissive medium (5 mg/mL Insulin, 0.1 mM Putrescine, 100 mg/mL Transferrin, 2.10⁻⁸M Progesterone (Sigma-Aldrich, Paris, France), 50 mg/mL Penicillin-Streptomycin (Invitrogen Life Technologies, Thermo Fisher Scientific, Waltham, Mass. USA) and growth factors including 10 ng/mL basic fibroblast growth factor (bFGF, Sigma-Aldrich), 20 ng/mL epidermal growth factor (EGF, R&D systems, Minneapolis, Minn., USA) and B27 (Invitrogen Life Technologies)). After 4 days, cells are trypsinized, suspended in HBSS buffer containing blocking solution (Miltenyi Biotec, Paris, France) and incubated with A2B5-APC and CD133-PE antibodies (Miltenyi Biotec) for 10 minutes. Cells are then fixed with 10% paraformaldehyde for 20 minutes and analyzed using flow cytometry (FACS Calibur™, BD Biosciences, San Jose, Calif., USA). A total of 100′000 events per sample are acquired with CellQuest Pro Software (BD Biosciences) and the data analyzed using FlowJo™ software and the Dean-Jet-Fox model analysis. Alternatively, the sphere formation assay can also be used to identify CSCs, since CSCs have the capabilities to grow and form spheres in vitro. For this, cells are plated at a density of 30′000 cells/25 cm³ flask in 3.5 mL of CSC permissive medium and maintained in a 5% CO₂/95% O₂ atmosphere. The number and size of spheres are assessed twice a week using 10× magnification and a calibrated micrometer reticule.

Alternatively brain CSCs can be identified in a sample by re-suspending brain tumor cells in DMEM-FCS 10% and incubating with anti-A2B5 mouse IgM antibody (cell supernatant 1/2 diluted, ATCC, Manassas, Va., USA) for 30 minutes at 4° C.; followed by washing and incubation with a magnetic microbead-tagged mouse-specific IgM rat antibody for 30 minutes at 4° C. Positive magnetic cell separation (MACS Miltenyi Biotec, Paris, France) can be performed using a MACS column with an average purity of 93% (ranging from 85% to 98%) as assessed by A2B5 control-immunostaining.

Alternatively, brain CSCs can be separated from a sample based on the binding of fluorescence-labelled antibodies to CSC markers, e.g. anti-A2B5 and anti-CD133 antibodies, on the cells using a FACS (fluorescence activated cell sorter) from any commercially available source (e.g. Bio-Rad [Hercules, Calif., USA] Benchtop S3™ cell sorter; Beckman Coulter [Brea, Calif., USA] MoFlo™ cell sorter; BD Biosciences FACSDiva™ cell sorter).

Sample Comparison

The subject according to the invention may be a human or animal in need of treatment. Preferably the subject is human.

The biomarker EB1 is measured ex vivo in a sample or samples taken from the human or animal body, preferably taken from the human body. The sample or samples are pre-obtained from the human or animal body, preferably pre-obtained from the human body before the sample is subjected to the method steps involving measuring the level of the biomarker.

A biomarker is in general a substance that is used as an indicator of a biological response, preferably as an indicator of the susceptibility to a given treatment, which in the present application is treatment with a compound of formula I or a pharmaceutically acceptable derivative thereof.

It has been found herein that higher EB1 levels in CSCs predict responsiveness to the compounds of the invention, or put another way, lower levels of CSCs predict resistance to the compounds of the invention.

In one preferred embodiment, higher EB1 levels in the sample relative to a standard value or set of standard values predicts responsiveness. As used herein, an increase or relatively high or high or higher levels relative to a standard level or set of standard levels means the amount or concentration of the biomarker in a sample is detectably more in the sample relative to the standard level or set of standard levels. This encompasses at least an increase of, or higher level of, about 1% relative to the standard, preferably at least an increase of about 5% relative to the standard. More preferably it is an increase of, or higher level of, at least about 10% relative to the standard. More particularly preferably it is an increase of, or higher level of, at least about 20% relative to the standard. For example, such an increase of, or higher level of, may include, but is not limited to, at least about 1%, about 10%, about 20%, about 30%, about 50%, about 70%, about 80%, about 90% or about a 100% or >100% increase relative to the standard.

In one preferred embodiment, lower EB1 levels in the sample relative to a standard value or set of standard values predicts resistance. As used herein, a decrease or relatively low or low or lower levels relative to a standard level or set of standard levels means the amount or concentration of the biomarker in a sample is detectably less in the sample relative to the standard level or set of standard levels. This encompasses at least a decrease of, or lower level of, about 1% relative to the standard, preferably at least a decrease of about 5% relative to the standard. More preferably it is a decrease of, or lower level of, at least about 10% relative to the standard. More particularly preferably it is a decrease of, or lower level of, at least about 20% relative to the standard. For example, such a decrease of, or lower level of, may include, but is not limited to, at least about 1%, about 10%, about 20%, about 30%, about 50%, about 70%, about 80%, about 90% or about a 100% decrease relative to the standard. Thus a decrease also includes the absence of detectable EB1 in the sample.

Preferably a higher level of EB1 in the sample

i) relative to a standard value or set of standard values from subjects with the same tumor histotype; or

ii) taken after treatment initiation and compared to the sample taken from the same subject before treatment initiation; or

iii) relative to a standard value or set of standard values from normal cells, tissue or body fluid;

predicts sensitivity of the brain neoplasm, preferably the sensitivity of CSCs of the brain neoplasm, to the compound of formula I or pharmaceutically acceptable derivative thereof.

More preferably, higher EB1 levels in a sample

i) relative to a standard value or set of standard values from subjects with the same tumor histotype; or

ii) taken after treatment initiation and compared to a sample or samples taken from the same subject before treatment initiation;

predicts sensitivity of the brain neoplasm, preferably the sensitivity of CSCs of the brain neoplasm, to the compound of formula I or pharmaceutically acceptable derivative thereof.

Especially preferably, higher EB1 levels in a sample or samples relative to a standard value or set of standard values from subjects with the same tumor histotype are predictive of sensitivity.

In one preferred embodiment, for the case i) where the measurement is compared in a sample or samples relative to a standard value or set of standard values from samples from subjects with the same tumor histotype as the sample to which it is to be compared, the standard value or set of standard values are established from samples from a population of subjects with that cancer type. The samples from these standard subjects may for example be derived from tumor tissue or from circulating tumor cells or from CSCs, as long as the origin of the sample is consistent between the standard and the sample to be compared.

In another preferred embodiment, for the case ii) where the measurement is compared in a sample or samples taken after treatment initiation and compared to a sample or samples taken from the same subject before treatment initiation, it is measured preferably to predict acquired resistance. The samples are compared to cells or tissue from the same biological origin. The prediction of acquired resistance would then indicate that the treatment with the compound should be discontinued. The biomarker is thus used to monitor whether further treatment with the compound is likely to give the required response (e.g. reduction of abnormal cells), or whether the cells have become non-responsive or resistant to such treatment.

In yet another preferred embodiment, for the case iii) where the measurement is compared in a sample or samples relative to a standard value or set of standard values from normal cells, tissue or body fluid, the standard value or set of standard values may be established from a sample of normal (e.g. non-tumorous) cells, tissue or body fluid. Such data may be gathered from a population of subjects in order to develop the standard value or set of standard values.

The standard value or set of standard values are established ex-vivo from pre-obtained samples which may be from cell lines or animal tumor models, or preferably biological material from at least one human subject and more preferably from an average of subjects (e.g., n=2 to 1000 or more).

The standard value or set of standard values may then be correlated with the response data of the same cell lines, or same subjects, to treatment with a compound of formula I or a pharmaceutically acceptable derivative thereof. From this correlation a comparator module, for example in the form of a relative scale or scoring system, optionally including cut-off or threshold values, can be established which indicates the levels of biomarker associated with a spectrum of response levels to the compound of formula I or a pharmaceutically acceptable derivative thereof. The spectrum of response levels may comprise relative sensitivity to the therapeutic activity of the compound, (e.g. high sensitivity to low sensitivity), as well as resistance to the therapeutic activity. In a preferred embodiment this comparator module comprises a cut-off value or set of values which predicts sensitivity to treatment.

For example, if an immunohistochemical method is used to measure the level of EB1 in a sample, standard values may be in the form of a scoring system. Such a system might take into account the percentage of cells in which staining for EB1 is present. The system may also take into account the relative intensity of staining in the individual cells. The standard values or set of standard values of the level of EB1 may then be correlated with data indicating the response, especially sensitivity, of the subject or tissue or cell line to the therapeutic activity of a compound of formula I or a pharmaceutically acceptable derivative thereof. Such data may then form part of a comparator module.

Response is the reaction of the cell lines, or preferably of the subject, or more preferably of the disease in a subject, to the activity, preferably therapeutic activity, of a compound of formula I or a pharmaceutically acceptable derivative thereof. The spectrum of response levels may comprise relative sensitivity to the activity, preferably therapeutic activity, of the compound, (e.g. high sensitivity to low sensitivity), as well as resistance to the activity, preferably therapeutic activity. The response data may for example be monitored in terms of: objective response rates, time to disease progression, progression free survival, and overall survival.

The response of a cancerous disease may be evaluated by using criteria well known to a person in the field of cancer treatment, for example but not restricted to,

Response Evaluation Criteria in Solid Tumors (RECIST) Guidelines, Source: Eisenhauer E A, Therasse P, Bogaerts J, Schwartz L H, Sargent D, Ford R, Dancey J, Arbuck S, Gwyther S, Mooney M, Rubinstein L, Shankar L, Dodd L, Kaplan R, Lacombe D, Verweij J. New response evaluation criteria in solid tumors: revised RECIST guideline (version 1.1). Eur J Cancer. 2009; 45:228-47;

RANO Criteria for High-Grade Gliomas, Source: Wen P Y, Macdonald D R, Reardon D A, Cloughesy T F, Sorensen A G, Galanis E, Degroot J, Wick W, Gilbert M R, Lassman A B, Tsien C, Mikkelsen T, Wong E T, Chamberlain M C, Stupp R, Lamborn K R, Vogelbaum M A, van den Bent M J, Chang S M. Updated response assessment criteria for high-grade gliomas: response assessment in neuro-oncology working group. J Clin Oncol. 2010; 28(11):1963-72;

CA-125 Rustin Criteria for Ovarian Cancer Response, Source: Rustin G J, Quinn M, Thigpen T, du Bois A, Pujade-Lauraine E, Jakobsen A, Eisenhauer E, Sagae S, Greven K, Vergote I, Cervantes A, Vermorken J. Re: New guidelines to evaluate the response to treatment in solid tumors (ovarian cancer). J Natl Cancer Inst. 2004; 96(6):487-8; and

PSA Working Group 2 Criteria for Prostate Cancer Response, Source: Scher H I, Halabi S, Tannock I, Morris M, Sternberg C N, Carducci M A, Eisenberger M A, Higano C, Bubley G J, Dreicer R, Petrylak D, Kantoff P, Basch E, Kelly W K, Figg W D, Small E J, Beer T M, Wilding G, Martin A, Hussain M; Prostate Cancer Clinical Trials Working Group. Design and end points of clinical trials for patients with progressive prostate cancer and castrate levels of testosterone: recommendations of the Prostate Cancer Clinical Trials Working Group. J Clin Oncol. 2008; 26(7):1148-59.

Sensitivity is associated with there being an observable and/or measurable reduction in, or presence of, one or more of the following: reduction in the number of abnormal cells, preferably cancerous cells or absence of the abnormal cells, preferably cancerous cells; for cancerous diseases: reduction in tumor size; inhibition (i.e., slowed to some extent and preferably stopped) of further tumor growth; reduction in the levels of tumor markers such as PSA and CA-125, inhibition (i.e., slowed to some extent and preferably stopped) of cancer cell infiltration into other organs (including the spread of cancer into soft tissue and bone); inhibition (i.e., slowed to some extent and preferably stopped) of tumor metastasis; alleviation of one or more of the symptoms associated with the specific cancer; and reduced morbidity and mortality.

In a preferred embodiment sensitivity means there is an observable and/or measurable reduction in, or absence of, one or more of the following criteria: reduction in tumor size; inhibition of further tumor growth, inhibition of cancer cell infiltration into other organs; and inhibition of tumor metastasis.

In a more preferred embodiment sensitivity refers to one or more of the following criteria: a reduction in tumor size; inhibition of further tumor growth, inhibition of cancer cell infiltration into other organs; and inhibition of tumor metastasis.

Measurement of the aforementioned sensitivity criteria is according to clinical guidelines well known to a person in the field of cancer treatment, such as those listed above for measuring the response of a cancerous disease.

Response may also be established in vitro by assessing cell proliferation and/or cell death. For example, effects on cell death or proliferation may be assessed in vitro by one or more of the following well established assays: A) Nuclear staining with Hoechst 33342 dye providing information about nuclear morphology and DNA fragmentation which are hallmarks of apoptosis. B) AnnexinV binding assay which reflects the phosphatidylserine content of the outer lipid bilayer of the plasma membrane. This event is considered an early hallmark of apoptosis. C) TUNEL assay (Terminal deoxynucleotidyl transferase mediated dUTP Nick End Labeling assay), a fluorescence method for evaluating cells undergoing apoptosis or necrosis by measuring DNA fragmentation by labeling the terminal end of nucleic acids. D) MTS proliferation assay measuring the metabolic activity of cells. Viable cells are metabolically active whereas cells with a compromised respiratory chain show a reduced activity in this test. E) Crystal violet staining assay, where effects on cell number are monitored through direct staining of cellular components. F) Proliferation assay monitoring DNA synthesis through incorporation of bromodeoxyuridine (BrdU). Inhibitory effects on growth/proliferation can be directly determined. G) YO-PRO assay which involves a membrane impermeable, fluorescent, monomeric cyanine, nucleic acid stain, which permits analysis of dying (e.g. apoptotic) cells without interfering with cell viability. Overall effects on cell number can also be analysed after cell permeabilisation. H) Propidium iodide staining for cell cycle distribution which shows alterations in distribution among the different phases of the cell cycle. Cell cycle arresting points can be determined. I) Anchorage-independent growth assays, such as colony outgrowth assays which assess the ability of single cell suspensions to grow into colonies in soft agar.

In one preferred embodiment relating to determination in vitro, sensitivity means there is a decrease in the proliferation rate of abnormal cells and/or reduction in the number of abnormal cells. More preferably sensitivity means there is a decrease in the proliferation rate of cancerous cells and/or a reduction in the number of cancerous cells. The reduction in the number of abnormal, preferably cancerous, cells may occur through a variety of programmed and non-programmed cell death mechanisms. Apoptosis, caspase-independent programmed cell death and autophagic cell death are examples of programmed cell death. However the cell death criteria involved in embodiments of the invention is not to be taken as limited to any one cell death mechanism.

In a preferred embodiment relating to determination of resistance in vitro, resistance means there is no decrease in the proliferation rate of abnormal cells and/or reduction in the number of abnormal cells. More preferably resistance means there is no decrease in the proliferation rate of cancerous cells and/or no reduction in the number of cancerous cells. The reduction in the number of abnormal, preferably cancerous, cells may occur through a variety of programmed and non-programmed cell death mechanisms. Apoptosis, caspase-independent programmed cell death and autophagic cell death are examples of programmed cell death. However the cell death criteria involved in embodiments of the invention is not to be taken as limited to any one cell death mechanism.

EB1

The term EB1 is used herein to encompass all the previously mentioned synonyms and refers to this entity on both the nucleic acid and protein levels as appropriate. Nucleic acid levels refer to for example mRNA, cDNA or DNA and the term protein includes the translated polypeptide or protein sequence and post-translationally modified forms thereof.

A preferred example of the protein sequence of EB1 (human EB1) is listed in SEQ. ID NO: 1. However, the term EB1 also encompasses homologues, mutant forms, allelic variants, isotypes, splice variants and equivalents of this sequence. Preferably also it encompasses human homologues, mutant forms, allelic variants, isotypes, splice variants and equivalents of this sequence. More preferably it encompasses sequences having at least about 75% identity, especially preferably at least about 85% identity, particularly preferably at least about 95% identity, and more particularly preferably about 99% identity, to said sequence.

In an especially preferred embodiment, EB1 is the entity on the nucleic acid or protein levels, which is represented on the protein level by SEQ ID NO: 1 or sequences having at least 95% identity with this sequence, preferably at least 99% identity. In a particularly preferred embodiment, EB1 is represented by SEQ. ID. NO:

A preferred example of the cDNA nucleic acid sequence of EB1 (Human EB1) is accessible via NCBI Reference Sequence U24166 represented by SEQ ID NO: 2. However, the term EB1 also encompasses modifications, more degenerate variants of said sequence, complements of said sequence, and oligonucleotides that hybridise to one of said sequences. Such modifications include, but are not limited to, mutations, insertions, deletions, and substitutions of one or more nucleotides. More preferably it encompasses sequences having at least about 75% identity to said sequence, especially preferably at least about 85% identity, particularly preferably at least about 95% identity and more particularly preferably about 99% identity.

In yet another preferred embodiment, EB1 is the entity on the nucleic acid or protein levels, which is represented on the nucleic acid level by SEQ ID NO. 2 or sequences having at least 95% identity with this sequence, preferably at least 99% identity. In a particularly preferred embodiment, EB1 is represented by SEQ. ID. NO: 2.

Level of EB1

Levels of EB1 may be detected either on the protein level or on the nucleic acid level, e.g. RNA or cDNA. The level of EB1 may be assayed in the sample by technical means well known to a skilled person. It may be assayed at the transcriptional or translational level.

In one preferred embodiment the level of EB1 nucleic acid, preferably EB1 mRNA, in a sample is measured. Examples of methods of gene expression analysis known in the art which are suitable to measure the level of EB1 at the nucleic acid level include, but are not limited to, i) using a labelled probe that is capable of hybridising to mRNA; ii) using PCR involving one or more primers based on the EB1 gene sequence, for example using quantitative PCR methods using labelled probes, e.g. fluorogenic probes, such as quantitative real-time PCR; iii) micro-arrays; IV) northern blotting V) serial analysis of gene expression (SAGE), READS (restriction enzyme amplification of digested cDNAs), differential display and measuring microRNA.

In a preferred embodiment the level of EB1 at the protein level is measured. Examples of methods of protein expression analysis known in the art which are suitable to measure the level of EB1 at the protein level include, but are not limited to, i) immunohistochemistry (IHC) analysis, ii) western blotting iii) immunoprecipitation iv) enzyme linked immunosorbant assay (ELISA) v) radioimmunoassay vi) Fluorescence activated cell sorting (FACS) vii) mass spectrometry, including matrix assisted laser desorption/ionization (MALDI, e.g. MALDI-TOF) and surface enhanced laser desorption/ionization (SELDI, e.g. SELDI-TOF).

The antibodies involved in some of the above methods may be monoclonal or polyclonal antibodies, antibody fragments, and/or various types of synthetic antibodies, including chimeric antibodies, DARPS (designed Ankyrin repeat proteins) or DNA/RNA aptamers. The antibody may be labelled to enable it to be detected or capable of detection following reaction with one or more further species, for example using a secondary antibody that is labelled or capable of producing a detectable result. Antibodies specific to EB1 are available commercially, for example, from BD Biosciences and Cell Signaling Technology, Inc., or can be prepared via conventional antibody generation methods well known to a skilled person.

Preferred methods of protein analysis are flow cytometry (FACS), ELISA, fluorescence microscopy, mass spectrometry techniques, immunohistochemistry and western blotting, more preferably FACS, western blotting and immunohistochemistry. In FACS, fluorescently labelled antibodies or probes are used to bind to specific cellular proteins or antigens on whole cells (either fixed or native) in suspension where the intensity of the signal from the detectable label bound to the cellular antigen corresponds to the amount of protein expressed in the single cell and can be quantified. In fluorescence microscopy, fluorescently labelled antibodies or probes are used to bind to specific cellular proteins (antigens) where the protein amount and the positions can be detected and measured by the signal from the detectable label. In western blotting, also known as immunoblotting, labelled antibodies may be used to assess levels of protein, where the intensity of the signal from the detectable label corresponds to the amount of protein, and can be quantified for example by densitometry.

Immunohistochemistry again uses labelled antibodies or probes to detect the presence and relative amount of the biomarker. It can be used to assess the percentage of cells for which the biomarker is present. It can also be used to assess the localisation or relative amount of the biomarker in individual cells; the latter is seen as a function of the intensity of staining.

ELISA stands for enzyme linked immunosorbant assay, since it uses an enzyme linked to an antibody or antigen for the detection of a specific protein. ELISA is typically performed as follows (although other variations in methodology exist): a solid substrate such as a 96 well plate is coated with a primary antibody, which recognises the biomarker. The bound biomarker is then recognised by a secondary antibody specific for the biomarker. This may be directly joined to an enzyme or a third anti-immunoglobulin antibody may be used which is joined to an enzyme. A substrate is added and the enzyme catalyses a reaction, yielding a specific colour. By measuring the optical density of this colour, the presence and amount of the biomarker can be determined.

It is preferred that the level of EB1 is measured in the CSCs. This may be done by observing the level of EB1 in the CSCs in the sample directly, or by enriching the sample for the CSCs prior to measuring the level of EB1. For example, the level of EB1 may be measured directly in the CSCs using immunohistochemistry, FACS or immunofluorescence by staining with reagents visualising specifically CSCs and EB1 concomitantly or by first staining with a reagent that visualises the CSCs, and secondly with a reagent that visualises EB1.

Uses of Biomarker

In one preferred embodiment, the biomarker is used to predict inherent sensitivity of the disease in a subject to the compound of formula I or a pharmaceutically acceptable derivative thereof as defined above.

In another preferred embodiment, the biomarker is used to predict acquired resistance of the disease in a subject to the compound of formula I or a pharmaceutically acceptable derivative thereof as defined above.

The biomarker may be used to select subjects suffering or predisposed to suffering from a disease, preferably cancer, for treatment with a compound of formula I or a pharmaceutically acceptable derivative thereof as defined above. The levels of such a biomarker may be used to identify patients likely to respond or to not respond or to continue to respond or to not continue to respond to treatment with such agents. Stratification of patients may be made in order to avoid unnecessary treatment regimes. In particular the biomarker may be used to identify subjects from whom a sample or samples display a higher level of EB1, relative to a standard level or set of standard levels, whereupon such subjects may then be selected for treatment with the compound of formula I or a pharmaceutically acceptable derivative thereof as defined above.

The biomarker may also be used to assist in the determination of treatment regimes, regarding amounts and schedules of dosing. Additionally, the biomarker may be used to assist in the selection of a combination of drugs to be given to a subject, including a compound or compounds of formula I or a pharmaceutically acceptable derivative thereof, and another chemotherapeutic (cytotoxic) agent or agents. Furthermore, the biomarker may be used to assist in the determination of therapy strategies in a subject including whether a compound of formula I or a pharmaceutically acceptable derivative thereof is to be administered in combination with targeted therapy, endocrine therapy, biologics, radiotherapy, immunotherapy or surgical intervention, or a combination of these.

EB1 may also be used in combination with other biomarkers to predict the response to a compound of formula I or a pharmaceutically acceptable derivative thereof and to determine treatment regimes. It may furthermore be used in combination with chemo-sensitivity testing to predict sensitivity and to determine treatment regimes. Chemo-sensitivity testing involves directly applying a compound of formula I to cells taken from the subject, for example from a subject with haematological malignancies or accessible solid tumors, for example tumors which include, but are not restricted to, breast and head and neck cancers or melanomas, to determine the response of the cells to the compound.

Method of Treatment

The invention also involves in some aspects a method of treatment and EB1 for use in a method of treatment, wherein the level of EB1 is first established relative to a standard level or set of standard levels or pre-treatment initiation levels and then a compound of formula I or a pharmaceutically acceptable derivative thereof as defined above, is administered if the level of EB1 in said sample is higher than a standard value or set of standard values or has not decreased relative to pre-treatment initiation levels respectively. The compound of formula I or a pharmaceutically acceptable derivative thereof may be administered in a pharmaceutical composition, as is well known to a person skilled in the art. Suitable compositions and dosages are for example disclosed in WO 2004/103994 A1 pages 35-39, which are specifically incorporated by reference herein. Compositions for enteral administration, such as nasal, buccal, rectal or, especially, oral administration, and for parenteral administration, such as intravenous, intramuscular or subcutaneous administration, to warm-blooded animals, especially humans, are especially preferred. More particularly, compositions for intravenous or oral administration are preferred. In one embodiment compositions for oral administration are particularly preferred.

The compositions comprise the active ingredient and a pharmaceutically acceptable carrier. An example of a composition includes, but is not limited to, hard capsules containing 1 mg active ingredient, 98 mg of mannitol and 1 mg magnesium stearate, or 5 mg active ingredient, 94 mg mannitol and 1 mg magnesium stearate.

The invention also relates in one aspect to a method of treating a neoplastic or autoimmune disease, preferably cancer, by first increasing the level of EB1 in a subject that has a sample with a lower level of EB1 compared to a standard level or set of standard levels, or pre-treatment initiation levels, then treating the subject with a compound of formula I or a pharmaceutically acceptable derivative as defined above. The level of EB1 may be increased by direct or indirect chemical or genetic means. Examples of such methods are treatment with a drug that results in increased EB1 expression and targeted delivery of viral, plasmid or peptide constructs, or antibody or siRNA or antisense to upregulate the level of EB1. For example viral or plasmid constructs may be used to increase the expression of EB1 in the cell. The subject may then be treated with a compound of formula I or a pharmaceutically acceptable derivative thereof.

A compound of formula I or a pharmaceutically acceptable derivative thereof can be administered alone or in combination with one or more other therapeutic agents. Possible combination therapy may take the form of fixed combinations, or the administration of a compound of the invention and one or more other therapeutic agents which are staggered or given independently of one another, or the combined administration of fixed combinations and one or more other therapeutic agents.

A compound of formula I or a pharmaceutically acceptable derivative thereof can, besides or in addition, be administered especially for tumor therapy in combination with chemotherapy (cytotoxic therapy), targeted therapy, endocrine therapy, biologics, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemo-preventive therapy, for example in patients at risk.

Kit and Device

In one aspect the invention relates to a kit, and in another aspect to a device, for predicting the response, preferably of a disease in a subject, to a compound of formula I or a pharmaceutically acceptable derivative thereof as defined above, comprising reagents necessary for measuring the level of EB1 in a sample. Preferably, the reagents comprise a capture reagent comprising a detector for EB1 and a detector reagent.

The kit and device may also preferably comprise a comparator module which comprises a standard value or set of standard values to which the level of EB1 in the sample is compared. In a preferred embodiment, the comparator module is included in instructions for use of the kit. In another preferred embodiment the comparator module is in the form of a display device, for example a strip of colour or numerically coded material which is designed to be placed next to the readout of the sample measurement to indicate resistance levels. The standard value or set of standard values may be determined as described above.

In respect of EB1, the reagents are preferably antibodies or antibody fragments which selectively bind to EB1. Suitable samples are tissue, tumor tissue or CSC samples, sections of fixed and paraffin-embedded or frozen tissue, tumor tissue or CSC specimens, and blood, cerebrospinal fluid and other body liquid-derived samples (including circulating tumor cells and CSCs). Preferably, the reagents are in the form of one specific primary antibody which binds to EB1 and a secondary antibody which binds to the primary antibody, and which is itself labelled for detection. The primary antibody may also be labelled for direct detection. The kits or devices may optionally also contain a wash solution(s) that selectively allows retention of the bound biomarker to the capture reagent as compared with other biomarkers after washing. Such kits can then be used in ELISA, western blotting, flow cytometry (FACS), immunofluorescence microscopy, immunohistochemical or other immunochemical methods to detect the level of the biomarker. Non-antibody based specific probes may also be used to detect the level of EB1, e.g. drug affinity responsive target stability (DARTS) or aptamers.

The reagents may also in another preferred embodiment be those that are capable of measuring the level of EB1 nucleic acids in a sample. Suitable samples are tissue, tumor tissue or CSC samples, sections of fixed and paraffin-embedded or frozen tissue, tumor tissue or CSC specimens, and blood, cerebrospinal fluid and other body liquid-derived samples (including circulating tumor cells and CSCs). Preferably, the reagents comprise a labelled probe or primers for hybridization to EB1 nucleic acid in the sample. Suitable detection systems, either based on PCR amplification techniques or detection of labelled probes, allow quantification of EB1 nucleic acid in the sample. This can be done i) in-situ on the specimen itself, preferably in sections from paraffin-embedded or frozen specimens, ii) in extracts from tumor, tissue or blood- and cerebrospinal fluid-derived specimens (including circulating tumor cells and CSCs), where suitable reagents selectively enrich for nucleic acids. The kits or devices enable the measurement and quantification of i) the amount of hybridized labelled probes to the specimens in-situ or ii) the amount of primer-based amplification products by methods based on specific physico-chemical properties of the probes itself or the reporters attached to the primers.

Similarly, the kit and device may contain reagents which selectively bind to CSCs. Such reagents may target CSC markers as described above.

Furthermore the device may comprise imaging devices or measurement devices (for example, but not restricted to, measurement of fluorescence) which further process the measured signals and transfer them into a scale in a comparator module.

In the present specification the words “comprise” or “comprises” or “comprising” are to be understood as to imply the inclusion of a stated item or group of items, but not the exclusion of any other item or group of items.

### Experimental Methodology

Tumor Samples from Patients

Glioblastoma (GBM) samples (World Health Organization, grade IV) from patients who did not receive chemotherapy or radiotherapy before surgery were obtained after informed consent. Primary fresh GBM samples were collected after surgery, in Dulbecco's modified Eagle's medium (DMEM) (Life technologies, Saint Aubin, France) supplemented with 0.5% foetal calf serum (FCS), Gibco-Invitrogen, Cergy Pontoise), 1% penicillin-streptomycin and 1% sodium pyruvate (Gibco-Invitrogen). Within 4 hours, tumors were washed, dissected, automatically sectioned using a McIlwain tissue chopper and enzymatically dissociated with both 5 mg/mL of Trypsin (Sigma-Aldrich, Paris, France) and 200 U/mL of DNAse (Sigma-Aldrich) for 10 minutes at 37° C. The reaction was stopped by adding DMEM 10% FCS. The suspension was filtered and collected cells were centrifuged. Cells were then counted and trypan blue staining (Sigma-Aldrich) confirmed more than 80% cell viability. Cells were then resuspended in DMEM-FCS 10% until their separation based on A2B5+ antigen expression.

GBM CSC Separation Based on A2B5+ Antigen Expression

Cells isolated from tumors were resuspended in phosphate-buffered saline, 0.5% Bovine Serum Albumin (BSA; Sigma-Aldrich), 2 mM EDTA (Sigma-Aldrich) and incubated 10 minutes at 4° C. with blocking reagent (Miltenyi Biotec, Paris, France). Anti-A2B5 microbeads were then added and incubated 15 minutes at 4° C. Cells were washed and positive magnetic cell separation (MACS Miltenyi Biotec, Paris, France) was performed using a MACS column. The average purity was approximately 93% (ranging from 85% to 98%) as assessed by A2B5 control-immunostaining by fluorescence microscopy. A2B5+cells were isolated from two glioblastoma multiforme tumors originating from the subventricular zone (GBM6) and the cortex (GBM9).

Culture of GBM6 and GBM9 Cells

GBM6 or GBM9 cells were plated at the density of 30′000 cells/25 cm³ flask in 3.5 mL of DMEM/F12 medium (Life technologies) supplemented with hormones (5 mg/mL Insulin, 0.1 mM Putrescine, 100 mg/mL Transferrin, 2.10⁻⁸M Progesterone; all Sigma-Aldrich), 50 mg/mL Penicillin-Streptomycin (Life technologies) and growth factors including 10 ng/mL basic fibroblast growth factor (bFGF, Sigma-Aldrich), 20 ng/mL epidermal growth factor (EGF), (R&D systems, Lille, France) and B27 (Life technologies) as a stem cell-permissive medium and maintained in 5% CO₂/95% O₂. Cultures were fed twice a week and spheres dissociated every two weeks.

Cell Transfections

An shRNA plasmid that specifically knocked down human EB1 (NM_012325) and a negative shRNA control plasmid (Mission® non-target shRNA control vector) were obtained from Sigma-Aldrich. Cells were transfected using the Lipofectamine™ 2000 system (Life technologies). For establishing stable clones, the shRNA-transfected cells and related control clones were selected in culture medium containing 2 μg/mL puromycin (Sigma-Aldrich) 24 hour post-transfection.

Stable GFP cell lines were obtained after transfection with a peGFP-N3 vector (Clonetech, Saint Germain en Laye, France) using the Lipofectamine™ 2000 system (Invitrogen) and selection with 0.6 mg/mL geneticin (Life technologies).

EB1 Immunoblot Analysis

GBM CSCs were lysed in lysis buffer (Tris 50 mM pH 8.0, NaCl 250 mM, Triton-×100 1%, SDS 0.1% and a cocktail of protease and phosphatase inhibitors (all from Sigma-Aldrich)). Thirty μg of total protein lysate were loaded onto a 12% SDS-PAGE gel. A nitrocellulose membrane (Bio-Rad, Marnes la Coquette, France) was blocked with 5% milk (powder) in PBS (Life technologies) with 0.1%-Tween (Sigma-Aldrich) pH 7.4 for 1 hour, and then incubated in the same solution with a mouse anti-EB1 antibody (clone 5, BD Biosciences, Le pont de Claix, France) ( 1/1000) and a mouse α-tubulin antibody (clone DM1A, Sigma Aldrich). Membranes were then washed for 15 minutes in PBS-5% milk three times and then incubated with anti-mouse peroxidase-conjugated secondary antibodies (Jackson Immunoresearch, Baltimore, USA) for 1 hour followed by three 15 minute washes in PBS. The bound antibodies were then detected using a chemiluminescence detection kit (Millipore, Saint Quentin en Yvelines, France). Signals were recorded with G:BOX (Syngene/Ozyme, Saint Quentin en Yvelines, France) and quantification used Image J software.

Cytotoxic Assay

Cells (5000 cells/well) were seeded in previously poly-DL-ornithine (Sigma-Aldrich) coated 96-well plates (10 μg/mL) and allowed to grow for 24 hours before treatment with BAL27862. Growth inhibition of cells was measured after 72 hours by using a sulforhodamine B assay kit (Sigma-Aldrich). Cell density was analyzed using a microplate reader (Labsystems Multiscan, Thermo, Villebon sur Yvette, France) and analysis of EC50 were performed with GraphPad 5.0 statistical software (GraphPad Software, La Jolla, USA). At least three independent experiments were performed.

Clonogenic Survival Assay

Cells were grown in previously poly-DL-ornithine (Sigma-Aldrich) coated 6-well plates (1000 cells/well) and after treatment for 72 hours with BAL27862, incubated for up to 5 days. Colonies were stained with 1% Crystal-violet solution in 20% methanol (Sigma-Aldrich). The colonies with more than 50 cells were counted. At least three independent experiments were performed.

Cell Migration Assay

Stem cells (50′000) were poured on the upper side of a transwell migration chamber (0.8 μm filter, BD) in DMEM. The lower side of the chamber was filled with DMEM, supplemented with 10% FCS. Cells were allowed to transmigrate for 5 hours and then chambers were removed. Cells that did not migrate stayed on the upper part of the filter and were removed with a cotton stick; cells on the lower side of the filter were fixed with 1% glutaraldehyde (Sigma, Aldrich) and stained with 1% Crystal-violet solution in 20% methanol (Sigma-Aldrich). After washing and drying, pictures of six fields per condition were imaged at a magnification of 10× and transmigrated cells were counted.

FACS Analysis of GBM6 Brain Tumor CSCs

GBM6 cells were seeded at 300′000 cells/well into poly-DL-ornithine-coated 6-well plates (10 μg/mL)(Sigma-Aldrich), in stem cell-permissive medium (5 mg/mL Insulin, 0.1 mM Putrescine, 100 mg/mL Transferrin, 2.10⁻⁸M Progesterone, 50 mg/mL Penicillin-Streptomycin and growth factors including 10 ng/mL basic fibroblast growth factor (bFGF), 20 ng/mL epidermal growth factor (EGF) and B27). One day later, cells were treated with BAL27862 for 72 hours. Then, cells were trypsinized, suspended in Hanks' Balanced Salt Solution (HBSS) buffer (Life technologies) containing blocking solution (Miltenyi Biotec) and incubated with A2B5-APC (clone 105HB29, reference 130-093-582) and CD133-PE (clone 293C3, reference 130-090-853) antibodies (Miltenyi Biotec) for 10 minutes. Cells were then fixed with 10% paraformaldehyde (Sigma-Aldrich) for 20 minutes and analyzed using flow cytometry (FACS Calibur™ BD Biosciences). A total of 100′000 events were counted for each sample and data were recorded with the CellQuest Pro Software (BD Biosciences) and analyzed using FlowJo software (Tree Star Inc., San Carlos, Calif.) and the Dean-Jet-Fox model analysis.

Self-Renewal Capacity Determination

GBM6 cells were seeded in previously poly-DL-ornithine (Sigma-Aldrich) coated 6-well plates (150′000 cells/well). After 24 hours, cells were treated or not with BAL27862 for 72 hours. At the end of treatment, the supernatant was removed and cells were harvested, dissociated into single cells and plated in 96-well plates (1-5 cells/well). Eight days later, the spheres were counted under an inverted light microscope (Leica DMI 4000B) (Leica, Saint Jorioz, France). Three independent experiments were performed.

Orthotopic GBM6 Xenografts

Approximately 100′000 cells of GBM6 GFP sh0 or shEB1 cells were stereotactically injected into the subventricular zone (0.5 mm anterior to bregma, −1 mm lateral, and −2.1 mm deep to the cortex surface) of 6-week-old athymic Nude mice. Grafted mice were intravenously treated with 25 mg/kg BAL101553 (three times at 30, 33 and 36 days after glioma implantation) or with vehicle (control). Forty five days after implantation, three animals of each group were sacrificed for analysis of tumor volumes (by three-dimensional brain reconstructions) or for FACS analysis.

Three-Dimensional Brain Reconstructions

For analysis of tumor volumes, mice were anesthetized and perfused with 4% paraformaldehyde. Brains were removed and fixed in 4% paraformaldehyde for 2 days at 4° C., then rinsed and conserved in PBS until dissection. Brains were entirely cut into 50-100-μm-thick sections using Vibratome (Leica) following the sagittal axis. A total of 60 sections per brain were obtained. Mapping and analysis of GFP-injected cells and tumors were performed by using a Leica DMLB fluorescence microscope, a Leica DC300F digital camera, and the Leica FW4000 imaging software (Leica). Three-dimensional brain reconstructions were obtained using FreeD software (Andrey P., Free-D: an integrated environment for three-dimensional reconstruction from serial sections. Journal of Neuroscience Methods. 2005; 145(1-2):233-244). Briefly, digitalized brain sections including ventricles and corpus callosum, based on the Franklin and Paxinos atlas, were instrumented to localize EGFP cells within these sections. Sixty digitalized sagittal sections were used to build a whole mouse brain.

FACS Analysis of Brain Tumor Cancer Stem Cells from Orthotopic GBM6 Tumors

After anesthesia, animals were perfused with PBS, and brains were removed and stored in HBSS buffer containing 2% FBS. Cells were dissociated in a cocktail of enzymes (DNase I (Roche, Rotkreuz, Switzerland), Collagenase D (Roche) and V (Sigma-Aldrich) with GentleMACS dissociator (Miltenyi Biotech)). The reaction was stopped by adding DMEM containing 10% FCS. The suspension was filtered and cells were resuspended in 40% Percoll (Sigma-Aldrich). After centrifugation, myelin was removed and cells were resuspended in HBSS buffer containing 2% FCS. Cells were incubated in HBSS containing 2% FCS with A2B5-APC and CD133-PE antibodies for 10 minutes. Cells were then fixed with 10% paraformaldehyde for 20 minutes and analyzed using flow cytometry. A total of 100′000 events were counted for each sample and data were recorded with the CellQuest Pro Software and analyzed using FlowJo software choosing the Dean-Jet-Fox model analysis.

GBM10 Flank Tumor Extraction and Extracellular Vesicle (Including Exosomes) Isolation from Serum Derived from Tumor-Bearing Mice

Female athymic nude mice (athymic Ncr-nu/nu) bearing large (250-420 mg) GBM10 flank tumors were euthanized according to standard procedures and the maximum amount of blood was drawn by cardiac puncture into a silicone coated BD Microtainer® blood collection tube (BD Diagnostics, Sparks, Md., USA). Serum was separated from the blood following manufacturer's instructions and stored at −80° C. until use.

Serum samples were thawed in a water bath at 25° C., then centrifuged (30 minutes, 2000×g, 4° C. (Heraeus Primo R centrifuge with microliter rotor 45°; Thermo Scientific, Thermo Fisher Scientific, Waltham, Mass. USA) to remove cells and debris. Clarified serum from two mice was pooled and used for extracellular vesicle isolation using the Total Exosome Isolation Kit (Invitrogen) according to manufacturer's instructions. Pellets were suspended in 50 μL 2×SDS sample buffer (0.125 M Tris pH 6.8, 4% SDS, 20% glycerol) and protein concentration determined by Pierce™ 660 nm Protein Assay (Thermo Scientific) according to manufacturer's instructions. Samples were stored at −80° C. until use. Immediately after sacrifice, GBM10 flank tumors from the same mice were dissected using standard procedures, snap frozen in liquid nitrogen and stored at −80° C. until use. Frozen tumors were placed in a gentleMACS M Tube (Miltenyi Biotec) containing 1 mL extraction buffer (50 mM Hepes pH 7.4, 150 mM NaCl, 5 mM EGTA, 1 mM EDTA, 1% NP-40, 1 mM DTT, 1 mM PMSF and 2× Protease and Phosphatase inhibitor cocktail [Thermo Scientific]) per 45 mg of tumor. Tumors were dissociated for 40 seconds using a gradient speed program in a gentleMACS Dissociator (Miltenyi Biotec). Tumor extracts were incubated on ice for 30 minutes and subsequently centrifuged at 10,000 rpm (Heraeus Primo R centrifuge with microliter rotor 45°; Thermo Scientific) for 30 minutes at 4° C. The supernatant was retained and protein concentration determined as above. Samples were stored at −80° C. until use.

GBM10 Flank Tumor and Extracellular Vesicle (Including Exosomes) Analysis

EB1 immunoblotting controls were prepared by siRNA transfection as follows: 1.2×10⁶ HeLa cells (ATCC, Manassas, Va., USA; reference CCL-2) were plated in a 75 cm² tissue culture flask for 7 hours before transfection. Cells were transiently transfected with 20 nM of non-targeting control (NTC) siRNA (Dharmacon GE Healthcare, Lafayette, Colo., USA; reference D-001810-10-20) or EB1 siRNA (Dharmacon GE Healthcare; reference L-006824-00-0005) using 1000 μL opti-MEM (Gibco) and 60 μL of HiperFect® Transfection reagent (QIAGEN, Venlo, Netherlands) according to manufacturer's instructions. Cells were cultured for a further 72 hours at 37° C. in a humidified atmosphere with 5% CO², prior to collection in 2×SDS sample buffer.

Extracellular vesicles derived from the serum of GBM10 tumor-bearing mice, control exosomes derived from human serum from healthy donors (HansaBioMed; reference HBM-PES-30/2) and HeLa cell lysates, prepared in 2×SDS sample buffer, were supplemented with 0.2 M DTT and 0.02% bromophenol blue for reducing conditions and 1× Protease and Phosphatase inhibitor cocktail (Thermo Scientific). For non-reducing conditions (required for CD9 immunoblotting only) the DTT was omitted. Tumor extracts were prepared by mixing three parts of tumor lysates with one part of 4× Laemmli sample buffer (Bio-Rad) and a 355 mM final concentration of 2-mercaptoethanol (Sigma). All samples were boiled for 5 minutes at 95° C.

Proteins (20 μg for tumor extracts, 10 μg for HeLa cell extracts and 30 μg or 10 μg for extracellular vesicles or control exosomes derived from human serum from healthy donors for EB1 or CD9 immunoblotting, respectively) were separated by SDS/PAGE and transferred to PVDF membranes (Trans-Blot® Turbo™, Bio-Rad). After blocking with 5% milk in PBS/0.1% (v/v) Tween-20 for 1 hour at room temperature, membranes were probed with the following primary antibodies at 4° C. overnight: anti-EB1 (Sigma; reference E3406), anti-CD9 (BioVision, Milpitas, Calif., USA; reference A1500-50) or anti-actin (Millipore, Merck KGaA, Darmstadt Germany; reference MAB1501). Membranes were incubated for 1 hour at room temperature with goat anti-rabbit-IgG-HRP secondary antibody (Jackson ImmunoResearch, West Grove, Pa., USA; reference 111-035-144) or goat anti-mouse IgG-HRP secondary antibody (Jackson ImmunoResearch; reference 115-035-146) and decorated proteins visualized using ECL Prime Western Blotting Detection Reagent (GE Healthcare, Chicago, Ill., USA). Signals were recorded using a FUSION SOLO S instrument and FUSION-CAPT software (Vilber Lourmat, MArne-la-Valée, France).

### DETAILED EXAMPLES

### Example 1: Comparison of Cytotoxic Activity of BAL27862 on GBM6 and GBM9 CSCs

Concentration-response curves of the cytotoxic activity of BAL27862 on GBM6 and GBM9 CSCs show that BAL27862 has comparable cytotoxic activity on both lines, with EC₅₀ of approximately 20 nM (see Table 1):

TABLE 1

| | EC₅₀ +/− SEM |
|---|---|
| GBM6 | 20.8 +/− 1.3 nM |
| GBM9 | 21.7 +/− 0.8 nM |

Data from at least three independent experiments.

### Example 2: EB1 Protein Expression Levels in GBM6 and GBM9 GBM CSCs

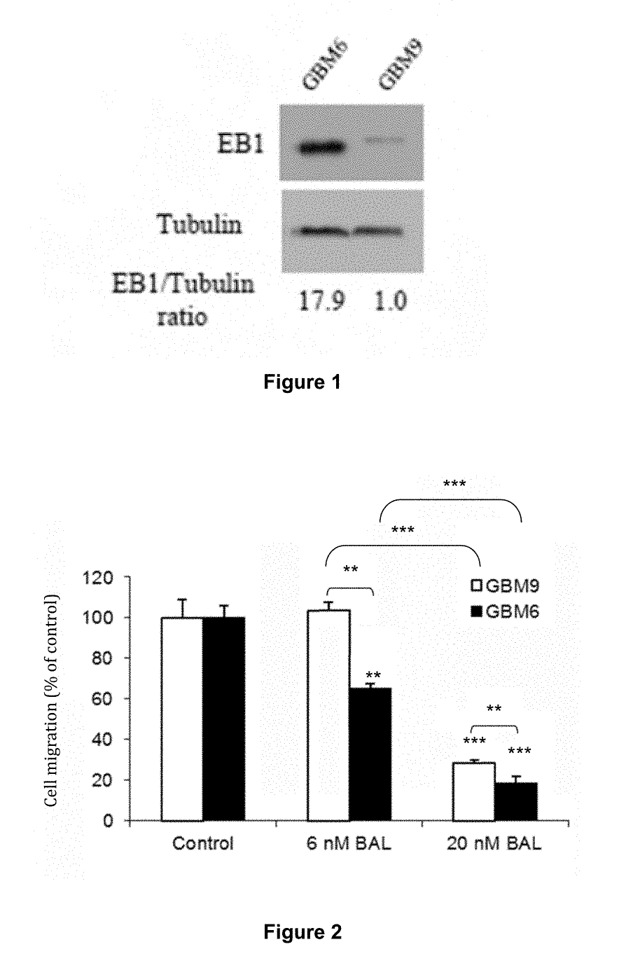

Immunoblot determination of EB1 expression levels in GBM6 versus GBM9 CSCs demonstrated that GBM6 cells express higher levels of EB1 protein than GBM9 (see FIG. 1). Normalizing to alpha-tubulin levels, GBM6 expressed 17.9 times higher amounts of EB1 protein than GBM9 cells.

### Example 3: BAL27862 Inhibits In Vitro Cell Migration More Efficiently in GBM6 than in GBM9 CSCs

Transwell migration assays of GBM6 and GBM9 cells were performed with two different BAL27862 concentrations, defined as non-cytotoxic (6 nM) and cytotoxic (20 nM) (see Example 1). As expected, the cytotoxic concentration (20 nM) drastically inhibited the migration of both cell lines to a similar degree, indicative of general cytotoxicity on the cells at this concentration, However, at the non-cytotoxic concentration of 6 nM, GBM6 cells showed a statistically significant inhibition of cell migration whereas GBM9 cells did not respond (see FIG. 2). This indicates that GBM6 migration can be specifically inhibited by low, non-cytotoxic concentrations of BAL27862, a phenomenon associated with higher EB1 levels as compared to GBM9.

### Example 4: Analysis of Stable GFP-Expressing GBM6 CSCs with EB1 Expression Levels Suppressed by shRNA Transfection

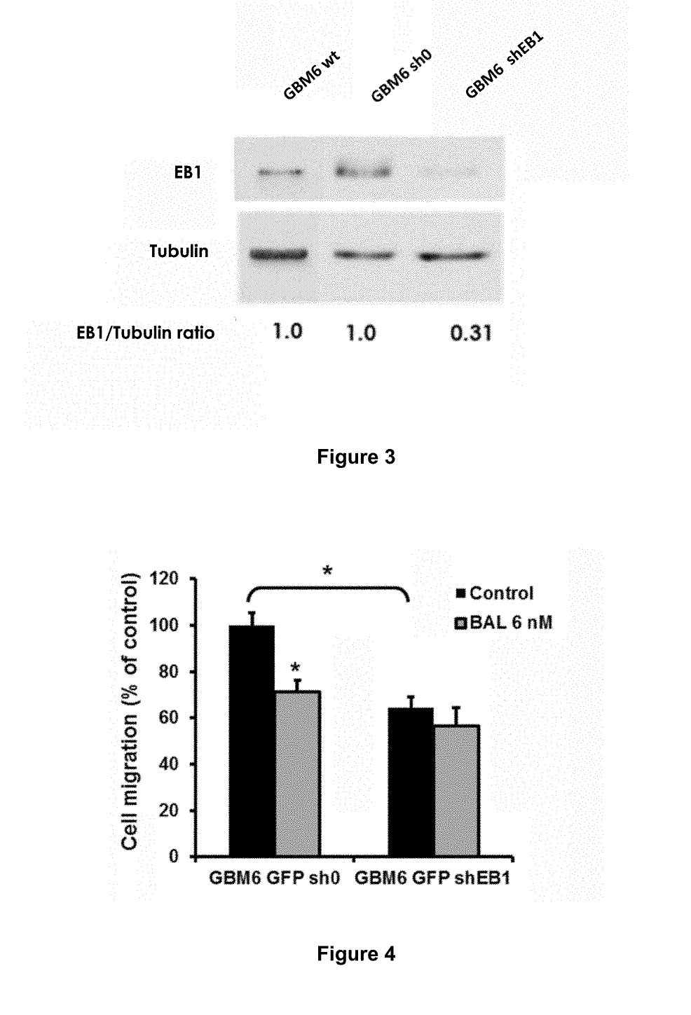

Stable GFP-expressing GBM6 cells transfected with shRNA (shEB1), non-targeted control shRNA (sh0) and non-treated GBM6 were tested for EB1 expression levels by immunoblotting (see FIG. 3). The measured signals were normalized to alpha-tubulin expression. GBM6 cells stably transfected with shEB1 show a 69% reduction of EB1 expression levels as compared to control GBM6 cells.

### Example 5: EB1 Downregulation Desensitizes GBM6 CSCs to the Anti-Migratory Effect of Non-Cytotoxic BAL27862 Concentrations

Cell migration measured at a BAL27862 concentration of 6 nM showed in the control EB1-expressing cells statistically significant inhibition of cell migration, whereas in the EB1 downregulated GBM6 cells the inhibitory effect of BAL27862 treatment was drastically reduced (see FIG. 4). This demonstrates that the EB1 protein is involved in the anti-migratory effect of BAL27862 in GBM CSCs.

### Example 6: BAL27862 Inhibits the Clonogenic Capacity of GBM6 CSCs in an EB1 Expression-Dependent Manner

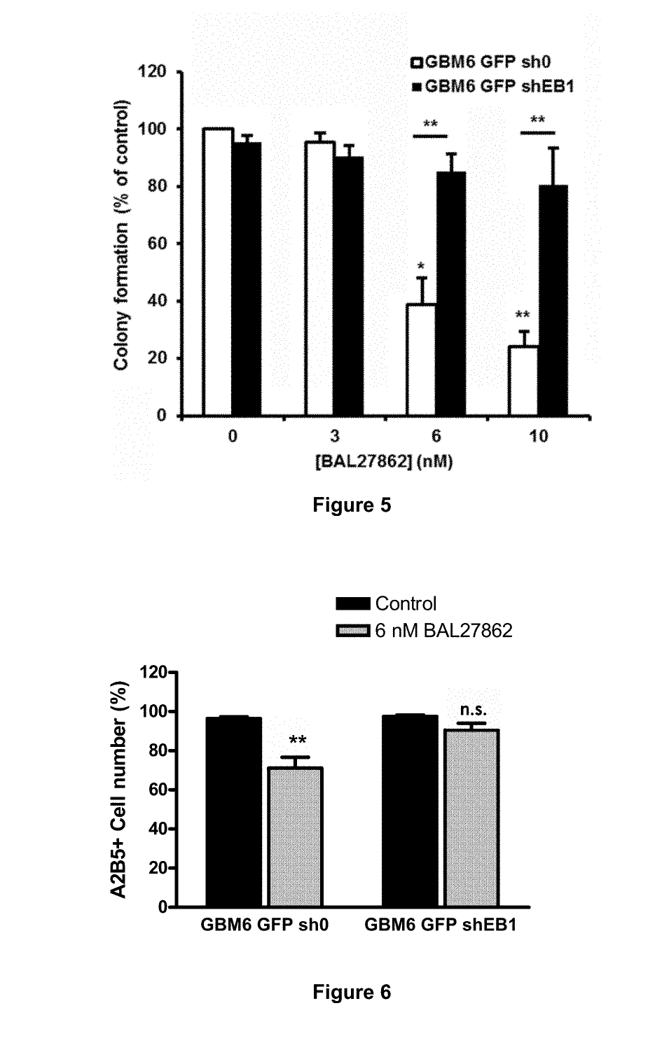

The colony formation capacity of GBM6 GFP sh0 (normal EB1 levels) and GBM6 GFP shEB1 (EB1 levels downregulated) was evaluated after 72 hours incubation with DMSO control or 3, 6 and 10 nM BAL27862 (non-cytotoxic concentrations) (see FIG. 5). Strikingly, BAL27862 impaired the clonogenic capacity of GBM6 CSCs at the sub-toxic doses of 6 and 10 nM in a statistically significant manner only in the EB1-expressing cells. This indicates that EB1 is required for the activity of BAL27862 in clonogenic assays.

### Example 7: BAL27862 Promotes Stem Cell Differentiation In Vitro in an EB1 Expression-Dependent Manner

FACs analysis of A2B5 positive GMB6 cell number after BAL27862 treatment showed a significant inhibition by 6 nM BAL27862 treatment only when EB1 was expressed (GBM6 GFP sh0 cells) (see FIG. 6). In contrast, GBM6 cells positive for A2B5 and negative for EB1 expression (GBM6 GFP shEB1, EB1 downregulated cells) were less sensitive to 6 nM BAL27862. Since loss of A2B5 expression is associated with differentiation, this indicates that BAL27862 induces differentiation of GBM6 CSCs in an EB1 expression-dependent manner.

### Example 8: BAL27862 Promotes Stem Cell Differentiation In Vitro in a EB1 Expression-Dependent Manner

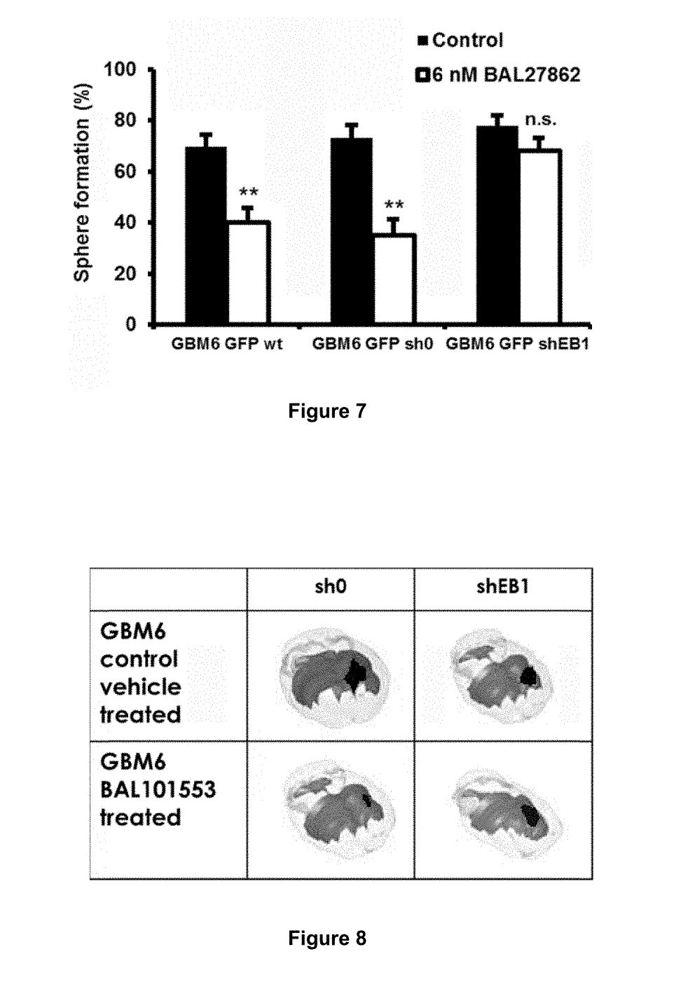

BAL27862 (6 nM) treatment interferes statistically significantly with sphere formation in a dose-dependent manner in GBM6 wild-type and GBM6 GFP sh0 cells (with normal EB1 levels) (see FIG. 7). However, EB1 downregulation (GBM6 GFP shEB1 cells) suppresses the inhibitory activity of BAL27862 in GBM6 CSCs. Since loss of capacity to form spheres is associated with differentiation, this indicates that BAL27862 induces differentiation of GBM6 CSCs in an EB1 expression-dependent manner.

### Example 9: BAL101553 (Prodrug of BAL27862) has Increased Activity on EB1-Expressing Orthotopic GBM6 Tumors

Mice receiving GBM6 GFP sh0 (normal EB1 expression levels) or GBM6 GFP shEB1 (EB1 downregulated) cells stereotactically implanted into the subventricular zone at day 0 were treated with 25 mg/kg BAL101553 i.v. or vehicle control at days 30/33/36 and sacrificed on day 45 for brain removal. Mouse brains were processed for serial sagittal sections which were then analysed and documented by the use of a fluorescence microscope. Pictures taken from single sections were used for a 3-dimensional reconstruction. BAL101553 treatment in comparison to vehicle treatment in EB1-expressing tumors resulted in a clear reduction of the tumor mass (size) and in tumor spreading, indicating a suppressive effect of BAL101553 on tumor growth and on tumor cell migration in vivo (see FIG. 8). However this effect of BAL101553 was minimal in EB1 negative tumors consistent with the in vitro data where EB1 expression positively correlates with BAL27862 activity on CSCs.

### Example 10: BAL101553 Treatment Reduces the Proportion of Undifferentiated CSCs in Orthotopic GBM6 Tumors

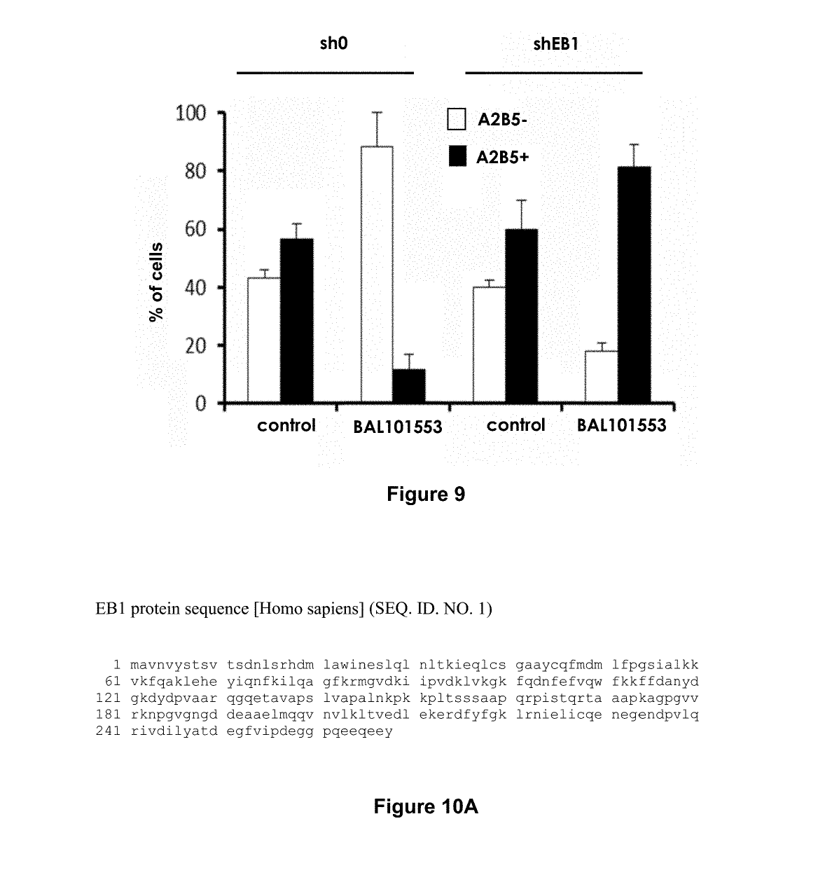

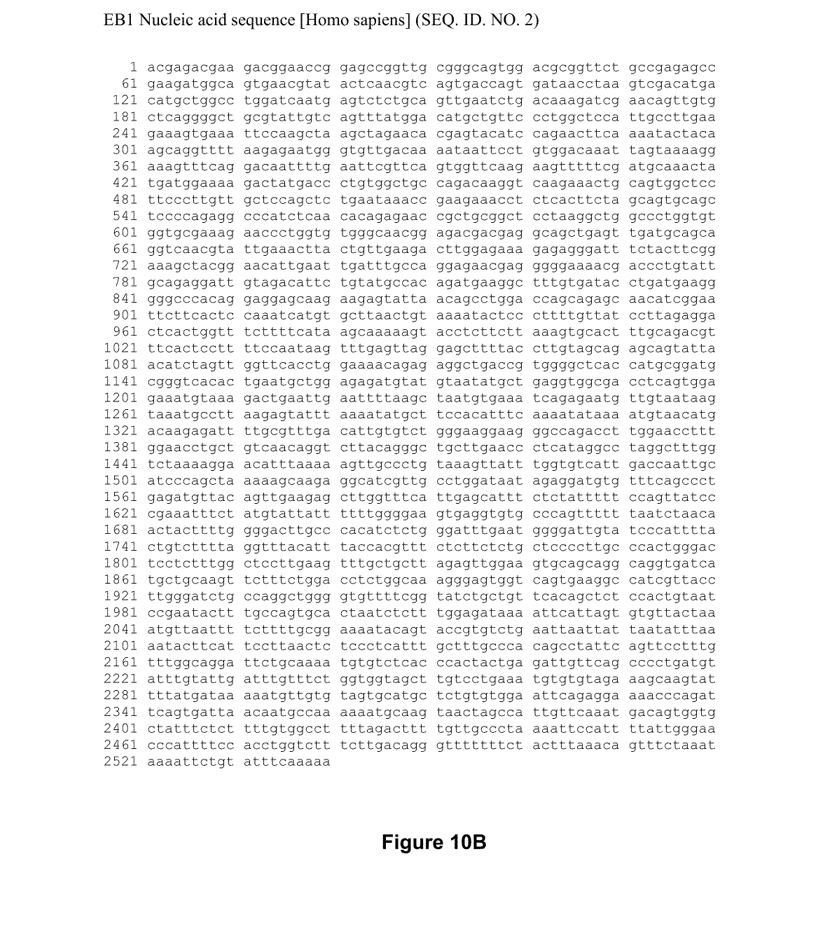

Mice receiving GBM6 GFP sh0 (normal EB1 expression levels) or GBM6 GFP shEB1 (EB1 downregulated) cells stereotactically implanted into the subventricular zone at day 0 were treated with 25 mg/kg BAL101553 i.v. or vehicle control at days 30/33/36 and sacrificed on day 45 for brain removal. Mouse brains were dissociated into single cell suspensions, stained for A2B5 positive GBM CSCs by using an anti-A2B5 antibody and analysed by FACS. The proportion of A2B5 negative to A2B5 positive GBM6 cells were calculated. BAL101553 treatment of EB1 expressing GBM6 tumors shifted the proportion of tumor cells strongly in favour of A2B5 negative cells (80% negative vs. 20% positive) which is a clear shift in comparison to the control (40% negative vs. 60% positive) (see FIG. 9). In contrast, in EB1 downregulated GBM6 tumors, the proportion of A2B5 positive cells rather increased upon BAL101553 treatment (approximately from 60% to 80%) indicating a higher content of undifferentiated GBM6 CSCs in EB1 downregulated brain tumors as compared to EB1-expressing tumors.

### Example 11: Extracellular Vesicles (Including Exosomes) Derived from EB1-Expressing GBM10 Tumor-Bearing Mice are Positive for EB1 Protein

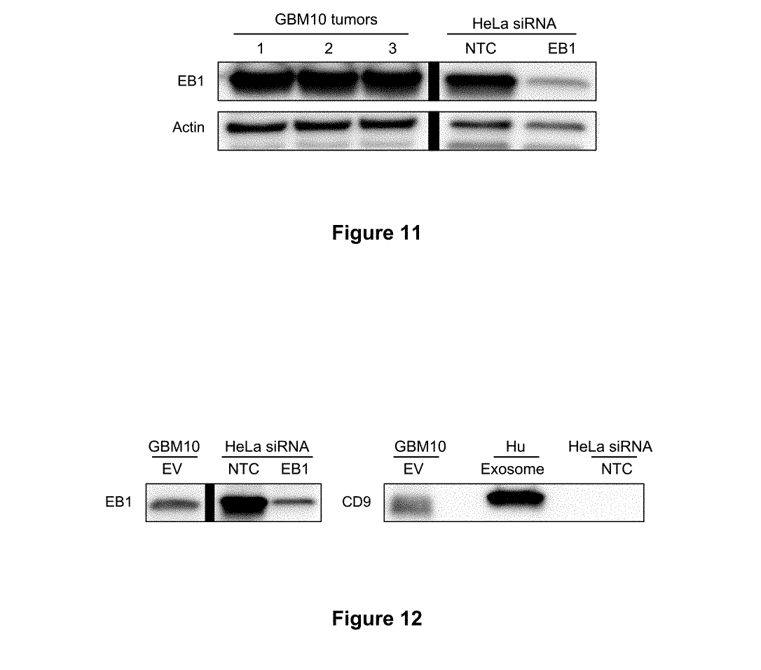

Tumors and extracellular vesicles isolated from mice bearing large GBM10 flank tumours were analysed by immunoblotting for EB1 expression. Efficient isolation of extracellular vesicles (including exosomes) was confirmed using control exosomes derived from human serum from healthy donors and CD9 as an extracellular vesicle (exosome) marker (Zarovni et al., Integrated isolation and quantitative analysis of exosome shuttled proteins and nucleic acids using immunocapture approaches. Methods, 2015; 87:46-58; Thery et al., Molecular Characterization of Dendritic Cell-derived Exosomes: Selective Accumulation of the Heat Shock Protein hsc73. The Journal of Cell Biology, 1999:3:599-610). Immunoblot of extracted tumors for EB1 expression showed consistent expression in three individual tumors (FIG. 11). The specificity of the antibody for EB1 was confirmed using HeLa extracts derived from cells treated with EB1 siRNA, which showed a dramatic decrease in EB1 as compared to control non-targeting siRNA control (NTC) extracts (FIG. 11). Moreover, extracellular vesicles (including exosomes) obtained from the serum of tumor-bearing mice were also shown to contain EB1 protein, indicating that this could be a useful surrogate source for GBM tumor EB1 expression analysis (FIG. 12).

1. A method for predicting the response of a brain neoplasm to a compound of formula I 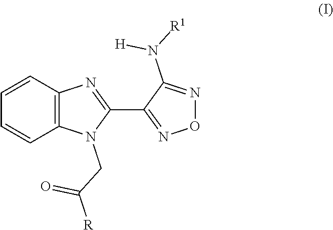
wherein
R represents phenyl or pyridinyl;
wherein phenyl is optionally substituted by one or two substituents independently selected from lower alkyl, lower alkoxy, amino, acetylamino, halogen and nitro;
and wherein pyridinyl is optionally substituted by amino or halogen;
R¹ represents hydrogen or cyano-lower alkyl;
or a pharmaceutically acceptable derivative thereof;
and wherein the prefix lower denotes a radical having up to and including a maximum of 4 carbon atoms,
comprising the step of determining the level of EB1 in a sample.

2. The method according to claim 1, wherein a higher level of EB1 in a sample taken from a subject relative to a standard value or set of standard values predicts sensitivity of the brain neoplasm to the compound of formula I or pharmaceutically acceptable derivative thereof.

3. The method according to claim 1, wherein a higher level of EB1 in cancer stem cells (CSCs) in a sample taken from a subject relative to a standard value or set of standard values predicts sensitivity of the brain neoplasm to the compound of formula I or pharmaceutically acceptable derivative thereof.

4. The method according to claim 1, wherein the response is of cancer stem cells (CSCs) of the brain neoplasm, preferably glioblastoma multiforme, to the compound of formula I or pharmaceutically acceptable derivative thereof.

5. The method according to claim 3, wherein the cancer stem cells (CSCs) express at least one cancer stem cell marker selected from the group consisting of ALDH1, CD24, CD44, CD90, CD133, Hedgehog-Gli activity, α6-integrin, ABCB5, β-catenin activity, CD26, CD29, CD166, LGR5, CD15, nestin, CD13, ABCG2, CD117, CD20, CD271, c-Met, CXCR4, Nodal-Activin, α2β1-integrin and Trop2, preferably selected from the group consisting of CD15, CD90, CD133, α6-integrin, nestin, and A2B5.

6. The method according to claim 3, wherein the cancer stem cells (CSCs) express the markers CD133 and/or A2B5.

7. The method according to claim 1, wherein the biomarker EB1 is measured ex vivo in a sample or samples taken from a subject.

8. The method according to claim 7, wherein the subject is a human.

9. The method according to claim 7, wherein the sample is derived from normal tissue, tumor tissue, cell lines, blood, cerebrospinal fluid, circulating tumor cells or cancer stem cells (CSCs),

10. The method according to claim 7, wherein the sample is derived from tumor tissue.

11. The method according to claim 7, wherein the cancer stem cells (CSCs) are identified in the sample and the level of EB1 is measured in the cancer stem cells (CSCs).

12. The method according to claim 1, wherein a higher level of EB1 in the sample
i) relative to a standard value or set of standard values from subjects with the same tumor histotype; or
ii) taken after treatment initiation and compared to the sample taken from the same subject before treatment initiation; or
iii) relative to a standard value or set of standard values from normal cells, tissue or body fluid;
predicts sensitivity of the brain neoplasm.

13. The method according to claim 12, wherein the sensitivity is sensitivity of cancer stem cells (CSCs) of the brain neoplasm, to the compound of formula I or pharmaceutically acceptable derivative thereof.

14. The method according to claim 1, wherein the brain neoplasm is selected from glial- and non-glial-tumors, astrocytomas, oligodendrogliomas, ependydomas, menigiomas, haemangioblastomas, acoustic neuromas, craniopharyngiomas, primary central nervous system lymphoma, germ cell tumors, pituitary tumors, pineal region tumors, primitive neuroectodermal tumors (PNETs), medullablastomas, haemangiopericytomas, spinal cord tumors including meningiomas, chordomas and genetically driven brain neoplasms, including neurofibromatosis, peripheral nerve sheath tumors and tuberous sclerosis.

15. The method according to claim 1, wherein the brain neoplasm is glioblastoma multiforme.

16. The method according to claim 1, wherein in the compound of formula I R, Y and R¹ are defined as follows:

| R | Y | R¹ |
|---|---|---|
| 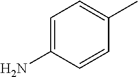 | O | CH₂CH₂CN |
| 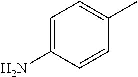 | O | H |
| 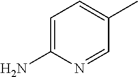 | O | CH₂CH₂CN |

or pharmaceutically acceptable derivatives thereof.

17. The method according to claim 1, wherein the compound is BAL27862 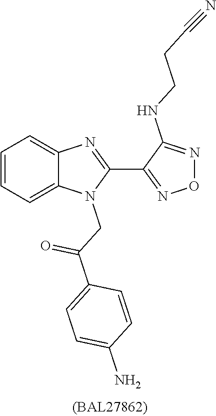 [SMILES: N#CCCNc1nonc1-c1nc2ccccc2n1CC(=O)c1ccc(N)cc1]
or pharmaceutically acceptable derivatives thereof.

18. The method according to claim 1, wherein the pharmaceutically acceptable derivative is selected from the group consisting of a salt, solvate, pro-drug, salt of a pro-drug of a compound of formula I.

19. The method according to claim 1, wherein the pharmaceutically acceptable derivative is a pro-drug and the pro-drug is an amide formed from an amino group present within the R group of the compound of formula I and the carboxy group of glycine, alanine or lysine.

20. The method according to claim 1, wherein the compound is BAL101553 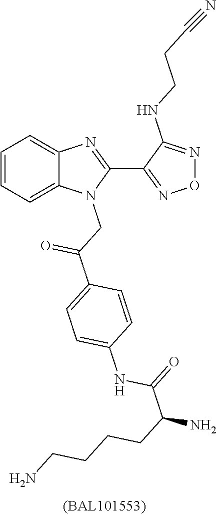 [SMILES: N#CCCNc1nonc1-c1nc2ccccc2n1CC(=O)c1ccc(NC(=O)[C@@H](N)CCCCN)cc1]
or a pharmaceutically acceptable salt thereof.

21. The method according to claim 20, wherein the pharmaceutically acceptable salt is a hydrochloride salt thereof, more preferably a dihydrochloride salt thereof.

22-23. (canceled)

24. A method of treating a brain neoplasm preferably glioblastoma multiforme, in a subject in need thereof, said method comprising
a) obtaining a sample of biologic material from the body of said subject;
b) determining the level of the EB1 in said sample; and
c) treating the subject with a compound of formula I as defined in claim 1, or a pharmaceutically acceptable derivative thereof, if the level of EB1 in said sample is higher than a standard value or set of standard values.

25. (canceled)

26. A kit for predicting the response of a brain neoplasm, preferably glioblastoma multiforme, to a compound of formula I or pharmaceutically acceptable derivative thereof, as defined in claim 1, comprising reagents necessary for measuring the level of EB1 in a sample and preferably further comprising a comparator module which comprises a standard value or set of standard values to which the level of EB1 in the sample is compared.

27. The kit according to claim 26, wherein the compound is as defined in claim 17 or claim 20.

28. The kit according to claim 26, wherein the reagents comprise a capture reagent comprising a detector for EB1 and a detector reagent, preferably wherein the capture reagent is an antibody;

29. The kit according to claim 26, wherein the kit comprises reagents necessary for measuring a level of EB1 in a sample and reagents necessary for identifying and/or capturing cancer stem cells (CSCs).

30. The kit according to claim 26, wherein the kit comprises BAL101553 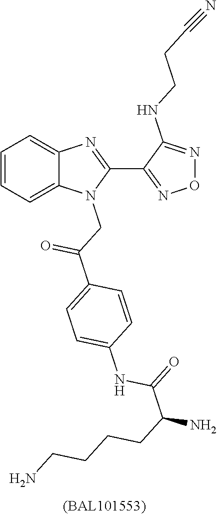 [SMILES: N#CCCNc1nonc1-c1nc2ccccc2n1CC(=O)c1ccc(NC(=O)[C@@H](N)CCCCN)cc1]
or a pharmaceutically acceptable salt thereof, in particular the dihydrochloride salt thereof.

31. A method for predicting the response to treatment of a brain neoplasm, preferably glioblastoma multiforme, in a subject by administration of a compound of formula I or pharmaceutically acceptable derivative thereof as defined in claim 1, said method comprising the steps of:
a) measuring the level of the EB1 in a sample obtained from the subject to obtain a value or values representing this level; and
b) comparing the value or values of the levels from step a) with a standard value or a set of standard values which comparison is predictive of responsiveness to the compound of formula I or pharmaceutically acceptable derivative thereof.

32. The method according to claim 31, wherein a higher level of EB1 in a sample taken from a subject relative to a standard value or set of standard values predicts sensitivity of the brain neoplasm to the compound of formula I or pharmaceutically acceptable derivative thereof.

33. The method according to claim 31, wherein the compound is BAL27862 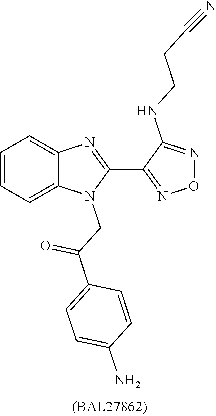 [SMILES: N#CCCNc1nonc1-c1nc2ccccc2n1CC(=O)c1ccc(N)cc1]
or pharmaceutically acceptable derivatives thereof, or BAL101553 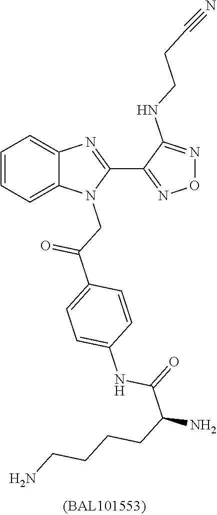 [SMILES: N#CCCNc1nonc1-c1nc2ccccc2n1CC(=O)c1ccc(NC(=O)[C@@H](N)CCCCN)cc1]
or a pharmaceutically acceptable salt thereof.

34-37. (canceled)

38. The method according to 1, wherein the sample is derived from a body fluid.

39. The method according to claim 1, wherein the sample is derived from blood.

40. The method according to claim 1, wherein the sample is derived from serum.

41. The method according to claim 1, wherein the determination of a higher level of EB1 is carried out by measuring the EB1 protein level or EB1 nucleic acid level.

42. The method according to 24, wherein the sample is derived from a body fluid.

43. The method according to claim 24, wherein the sample is derived from blood.

44. The method according to claim 24, wherein the sample is derived from serum.

45. The method according to claim 24, wherein the determination of a higher level of EB1 is carried out by measuring the EB1 protein level or EB1 nucleic acid level.

46. The kit according to 26, wherein the sample is derived from a body fluid.

47. The kit according to 26, wherein the sample is derived from blood.

48. The kit according to 26, wherein the sample is derived from serum.

49. The kit according to 26, wherein the determination of a higher level of EB1 is carried out by measuring the EB1 protein level or EB1 nucleic acid level.

50. The method according to 31, wherein the sample is derived from a body fluid.

51. The method according to claim 31, wherein the sample is derived from blood.

52. The method according to claim 31, wherein the sample is derived from serum.

53. The method according to claim 31, wherein the determination of a higher level of EB1 is carried out by measuring the EB1 protein level or EB1 nucleic acid level.
